(19) United States
(12) Patent Application Publication
(10) Pub. No.: US 2009/0312411 A1
(43) Pub. Date: Dec. 17, 2009

(54) PREVENTING OR REDUCING RISK OF MISCARRIAGES

(75) Inventors: Guillermina Giradi, New York, NY (US); Patricia Marie Redecha, Staten Island, NY (US)

Correspondence Address: DECHERT LLP, P.O. BOX 390460, MOUNTAIN VIEW, CA, 94039-0460, US

(73) Assignee: New York Society for the Raptured and Crippled Maintaining the Hospital for Special Surgery, New York, NY (US)

(21) Appl. No.: 12/368,229

(22) Filed: Feb. 9, 2009

Related U.S. Application Data
(60) Provisional application No. 61/040,119, filed on Mar. 27, 2008.

(51) Int. Cl. A61K 31/35; A61K 31/225; G01N 33/68; C12Q 1/02; A61P 15/06

(52) U.S. Cl. 514/460; 514/548; 436/86; 435/29

(57) ABSTRACT

The present disclosure provides methods of reducing pregnancy loss or miscarriage in patients by administering compounds that inhibit tissue factor (TF) expression and/or activity on neutrophils and/or monocytes, such as for example, statins, and methods of diagnosing patients having an increased risk of miscarrying.

### CROSS REFERENCES TO RELATED APPLICATIONS

This application claims benefit under 35 U.S.C. § 119(e) of application Ser. No. 61/040,119, filed Mar. 27, 2008, the contents of which are incorporated herein by reference.

### TECHNICAL FIELD

The inventions described herein relate to methods of preventing or reducing the risk of miscarriages, particularly in women who have suffered recurrent miscarriages, as well as to methods of diagnosing patients at risk of miscarrying.

### BACKGROUND

On average, 50% to 70% of all conceptions fail. The journey from conception to birth is fraught with danger. Complications that occur during pregnancy remain a serious clinical problem, and the triggers and mediators of placental and fetal damage are not completely understood. Recurrent pregnancy loss affects 1% to 3% of couples. In addition, preterm birth occurs in up to 10% of pregnancies, accounting for 70% of neonatal deaths and related neonatal morbidity, including neurological, respiratory, and metabolic complications in the newborn. A common condition associated with pregnancy loss is preeclampsia, which is a potentially life-threatening disease of women during pregnancy featuring hypertension, proteinuria and edema with variable coagulation and liver disorders. The condition occurs in about 5-7% of first pregnancies, usually after 20 weeks of gestation, and usually affects women at extremes of reproductive age. Preeclampsia is associated with substantial risks: for the fetus, these include intrauterine growth restriction, death and prematurity with attendant complications, whereas the mother is at risk for seizures (eclampsia), renal failure, pulmonary edema, stroke and death. Preeclampsia only occurs during pregnancy and its symptoms resolve after delivery. Factors from the placenta are thought to be involved, but the initiating cause of preeclampsia remains unknown.

The cost of caring for such conditions has been estimated at six billion dollars annually. Despite aggressive attempts to understand the basic biology underlying neonatal death and morbidity, their incidence has remained unchanged over the past three decades. Furthermore, in 50% to 60% of cases the well-established genetic, anatomic, endocrine, and infectious causes of fetal damage are not demonstrable. Thus, there is a need for methods to prevent or reduce the risk of miscarriages, especially recurrent miscarriages and preeclampsia, by safe and efficient methods, as well as methods of diagnosing patients at risk of miscarrying.

### SUMMARY

Up to 3% of women suffer recurrent miscarriages. Though the cause of recurrent miscarriages in most women is unknown, an immune mechanism, involving the inappropriate and subsequently injurious recognition of the embryo by the mother's immune system, has been proposed (American College of Obstetricians and Gynecologists, ACOG Practice Bulletin #24, ACOG, Washington, D.C., 2001; Clark et al., 2001, Hum Reprod Update 7:501-511; Mellor et al., 2001, Nat Immunol 2:64-8).

Indeed, 80% of the unexplained abortions are thought to be caused by an immune mechanism. Mammalian mothers are faced with a problem: the genome of the fetus they carry within their wombs is half maternal and half paternal. Fetuses express paternal antigens early in development. Thus, antigens presented by the fetus that are paternal in origin would be considered foreign by the mother's immune system (Billingham & Medawar, 1953, Nature 172:603-606). The maternal immune system may be prevented from recognizing the foreign fetal tissue and/or the maternal immune system may be prevented from developing an immune response in a successful pregnancy (Raghupathy, 2001, Immunol 13:219-227; Vince & Johnson, 1995, Human Reproduction 10: 107-113).

In a particular disorder, termed the antiphospholipid syndrome (“APS”), recurrent miscarriages are caused by the immune system's own production of anti-phospholipid antibodies (“aPL”). There is an association between aPL in the circulation and pregnancy loss, and between 3% and 7% of pregnant women have the antibodies. In low risk pregnancies, aPL are associated with a nine-fold increase in pregnancy loss, while in high risk pregnancies with at least three previous losses, they are associated with a 90% risk of further pregnancy loss.

Animal studies have shown the importance of inflammation in the pathogenesis of aPL-induced pregnancy loss (Holers et al., 2003, J Exp Med 195(2):211-20; Girardi et al., 2004, J Clin Invest 112(11): 1644-54). Recently, human studies showed that inflammation in the placenta may contribute to APS pregnancy complications, reinforcing this new concept of the antiphospholipid syndrome as an inflammatory disorder (Stone et al., 2006, Placenta 27(4-5):457-67).

It has also recently demonstrated that inflammation, specifically activation of complement with generation of the anaphylotoxin C5a, is crucial in fetal injury induced by aPL. TF expression in neutrophils (induced by C5a) contributes to respiratory burst and subsequent trophoblast injury and pregnancy loss induced by aPL (see, e.g., FIG. 1). It was found that either blockade of TF activity with an anti-TF monoclonal antibody in wild type mice or a genetic reduction of TF expression prevented aPL-induced inflammation and pregnancy loss (Redecha et al., 2007, Blood 110(7):2423-2431, the disclosure of which is incorporated herein by reference).

As will be discussed in more detail below, animal data presented herein demonstrate, for the first time, that compounds that inhibit expression of TF on neutrophils and/or monocytes reduce loss of pregnancy in two different murine models of miscarriage: a murine model of aPL-induced pregnancy loss and a murine model of pregnancy loss that is not aPL-dependent. While not intending to be bound by any theory of operation, and with reference to FIG. 1 and FIG. 13, these studies demonstrate that increased expression of TF on neutrophils and/or monocytes is associated with pregnancy loss, and that compounds capable of inhibiting TF expression provide a new and powerful means of reducing pregnancy loss or miscarriage, in both women who exhibit APS and women who have suffered miscarriages of unknown origin.

Accordingly, in one aspect, the present disclosure provides methods of inhibiting TF expression as a therapeutic approach towards preventing and/or reducing pregnancy loss or miscarriage, particularly in preventing and/or reducing the risk of preeclampsia or intrauterine growth restriction (IUGR). In some embodiments, the methods comprise administering to a woman who is either pregnant or planning to become pregnant an amount of a TF-expression inhibitor compound effective to prevent and/or reduce the likelihood of pregnancy loss or miscarriage, particularly to prevent and/or reduce the likelihood of preeclampsia or IUGR.

In some embodiments, the methods comprise administering to a woman who is pregnant or planning to become pregnant and has increased number of TF-positive neutrophils, increased number of TF-positive monocytes, increased level of TF in blood, and/or increased level of TF in chorionic villus (CV) as compared to those of normal pregnant women or normal non-pregnant women, respectively, an amount of a TF-expression inhibitor compound effective to prevent and/or reduce the likelihood of pregnancy loss or miscarriage, particularly to prevent and/or reduce the risk of preeclampsia or IUGR.

The TF-expression inhibitor compound reduces or inhibits expression of TF on neutrophils and/or monocytes, such as by inhibiting transcription of a gene encoding TF, thereby blocking synthesis of TF mRNA; inhibiting translation of mRNA encoding TF, thereby blocking synthesis of TF; or inhibiting one or more components affecting TF expression. TF expression inhibitor compounds may include, by way of example and not limitation, small molecules, antibodies, polypeptides and polynucleotides. As specific non-limiting examples, in some embodiments, compounds useful for inhibiting TF expression include antisense RNA, siRNA and miRNA oligonucleotides.

Statins are a group of compounds which are commonly used to reduce the level of cholesterol in the blood. They competitively inhibit 3-hydroxy-3-methylglutaryl-coenzyme A (“HMG-CoA”) reductase, the enzyme that catalyzes the rate-limiting step in cholesterol synthesis. Statins have been linked to a wide range of vascular benefits. In addition to lipid lowering, statins are postulated to exhibit pleiotropic properties such as inhibition of inflammation and coagulation (Takemoto et al., 2001, Arterioscler Thromb Vasc Biol. 21:1712-1719; Almog, 2004, Circulation 110:880-885). Simvastatin has been shown to reduce TF expression and activity in blood monocytes in patients with nephritic syndrome (Wei, 2007, Eur J Med Res 12(5):216-21). Other statins also suppress TF expression in different cell types (Pierangeli et al., 2005, J Thromb Haemost 3(5):1112-3; and Kunieda, 2003, Thromb Res 2003; 110(4):227-34).

Thus, an important class of TF-expression inhibitor compounds that can be used in the methods described herein is the statins, including by way of example and not limitation, atorvastatin, cerivastatin, fluvastatin, lovastatin, mevastatin, pravastatin, rosuvastatin, and simvastatin. The statin compounds can be in the form of free acids or bases, or in the form of pharmaceutically acceptable salts. In some embodiments, the TF expression inhibitor compound is simvastatin or a salt thereof. In some embodiments, the TF expression inhibitor compound is pravastatin or a salt thereof.

In certain embodiments, for instance where the patient has increased presence of TF but does not have anti-phospholipid antibodies associated with APS, a TF-activity inhibitor compound can be administered. TF activity inhibitor compounds are compounds that inhibit or reduces TF-VIIa signaling through PAR2, such as antibodies that bind to TF.

The TF-expression inhibitor compounds and TF-activity inhibitor compounds can be administered by any means suitable for the delivery of the specific being utilized. In the case of statins, oral dosage forms are available commercially that can be used in the methods described herein.

The TF expression inhibitor may be administered alone, or it may be administered in combination with one or more additional TF-expression inhibitor compounds and/or in combination with one or more additional therapeutic agents.

The TF-expression inhibitor compound and TF-activity inhibitor compounds can be administered to virtually any woman who is either pregnant or who is planning to become pregnant. As mentioned above, approximately 3% of woman in the U.S. suffer recurrent miscarriages. In many cases, the cause of the miscarriages is unknown. In some instances, such as in cases where the women exhibits or suffers from APS, the miscarriages are believed to be due to production of aPL. The animal data presented herein demonstrate that the TF-expression inhibitor compounds described herein are useful to reduce or prevent miscarriage in women, whether the miscarriages are associated with aPL or of unknown origin. In some embodiments, the TF expression inhibitor compounds are used to prevent or reduce miscarriage or pregnancy loss in a patient who exhibits or suffers from APS, has been previously diagnosed with APS, or has anti-phospholipid antibodies but no previous miscarriages. In some embodiments, the TF expression inhibitor compounds are used to prevent or reduce miscarriage or pregnancy loss in a woman who has suffered multiple recurrent miscarriages, whether of known or unknown origin. In some embodiments, the TF expression inhibitor compounds are used to prevent or reduce miscarriage or pregnancy loss in a woman experiencing a first pregnancy, or who has not previously miscarried.

In some embodiments, where assessment of TF levels is a basis for therapeutic intervention, the patient can be pregnant or is planning to become pregnant for the first time, has not had a previous pregnancy loss or miscarriage, has not suffered previous miscarriages, or has not manifested preeclampsia in a previous pregnancy; but has not manifested presence of anti-phospholipid antibodies associated with APS or has not been previously been diagnosed with APS.

In some embodiments, where assessment of TF levels is a basis for therapeutic invention, the patient can be pregnant or planning to become pregnant and has had a previous miscarriage, suffered recurrent miscarriages, or manifested preeclampsia in a previous pregnancy but has not manifested presence of anti-phospholipid antibodies associated with APS or has not been previously diagnosed with APS.

The TF expression inhibitor compound therapy can be initiated before pregnancy, for example, about one month in advance of a planned pregnancy, or after the patient has become pregnant. The therapy can be applied for a period considered as high risk for miscarriage, such as, the first trimester, and then discontinued, or it may be applied throughout the duration of pregnancy, up to child birth.

In another aspect, the present disclosure provides methods of screening or diagnosing patients to identify those at risk of having a miscarriage. The methods generally comprise analyzing neutrophils, monocytes, maternal blood, chorionic villus, or combinations thereof, from the patient for increased presence of TF. The methods can be practiced by assessing the levels of TF per se, for example by quantification of TF on neutrophils, monocytes, maternal blood, or chorionic villus, assessing TF-dependent neutrophil or monocyte activity, such as release of oxygen species and/or phagocytosis, and/or assessing TF activity, such as anti-coagulation activity. In general, TF expression on neutrophils, monocytes, and chorionic villus of normal, healthy women is relatively low. Any value over the numbers found in normal pregnant women can be considered indicative of risk, with higher values correlating with higher risk.

In the case of release of reactive oxygen species and phagocytosis, any values greater than 10% and 20%, respectively, can be indicative of risk of miscarriage.

Assays suitable for measuring TF expression on neutrophils and monocytes and release of reactive oxygen species and/or phagocytosis by neutrophils and monocytes are described in Redecha et al., 2007, Blood 110(7):2423-2431, the disclosure of which is incorporated by reference. Assays suitable for measuring TF in blood include, among others, coagulation assays and TF antibody based ELISAs.

### BRIEF DESCRIPTION OF THE FIGURES

FIG. 1 provides a cartoon illustrating antiphospholipid antibody-induced expression of tissue factor and its concomitant oxidative burst and tissue injury;

FIG. 2 provides a graph demonstrating that simvastatin prevents fetal loss in a murine model of antiphospholipid syndrome (APS);

FIG. 3 provides photographs of uteri from mice treated with anti-phospholipid antibodies alone (top panel) and in combination with simvastatin (bottom panel);

FIG. 4 provides a graph illustrating that aPL-induced TF and PAR-2 synthesis increase is blocked by simvastatin;

FIG. 5 provides photographs of decidual tissue illustrating that simvastatin decreases free-radical-mediated lipid peroxidation in the decidual tissue of a murine model of APS;

FIG. 6 provides a graph demonstrating that pravastatin prevents fetal loss in a murine model of antiphospholipid syndrome (APS);

FIG. 7 provides a graph illustrating that pravastatin prevents fetal loss in a murine model of miscarriage of unknown origin (non aPL-dependent);

FIG. 8 provides a graph illustrating that simvastatin prevents intrauterine growth restriction (IUGR) in a murine model of APS;

FIG. 9 provides a graph illustrating that pravastatin prevents intrauterine growth restriction (IUGR) in a murine model of APS;

FIG. 10 provides a graph illustrating that pravastatin prevents intrauterine growth restriction (IUGR) in a murine model of miscarriage of unknown origin (non aPL-dependent);

FIG. 11 provides a graph demonstrating that inhibition of the aPL-induced inflammation pathway, but not the coagulation cascade, prevents pregnancy loss in a murine model of APS;

FIG. 12 provides a graph illustrating that an antibody that blocks TF-VIIa signaling through PAR2, but not an antibody that blocks the coagulation cascade, prevented aPL-induced ROS and phagocytosis increase in mice;

FIG. 13 provides a cartoon illustrating the role of TF in a non-aPL induced pregnancy loss;

FIG. 14A provides photomicrographs of deciduas of abortion prone CBA/J×DBA/2 matings and control CBA/J×BALB/c matings stained for TF expression (see Example 12);

FIG. 14B provides results of staining for presence of TF in human placentas from IUGR neonates and neonates of normal body weight;

FIG. 14C provides placental perfusion studies with FITC labeled dextran, comparing the blood supply in placentas from CBA/J×DBA/2 mice and control CBA/J×BALB matings with normal pregnancies;

FIGS. 15A, 15B and 15C provide graphs showing the effects of treating CBA/J×DBA/2 mice with hirudin, fondaparinux, anti-TF antibody 1H1, and dichloromethylene diphosphonate (Cl2MDP) on fetal resorption, fetal weight, and litter size;

FIG. 15D provides decidual TF expression in CBA/J×DBA/2 mice lacking monocytes;

FIG. 15E provides FACs profile of TF-positive monocytes from CBA/J×DBA/2 mice as compared to TF-positive monocytes from control CBA/J×BALB/c mice, showing increased TF-positive monocytes in CBA/J×DBA/2 mice;

FIG. 16A provides results of analysis for STAT-8 and FIG. 16B provides detection of superoxide production in placentas of CBA/J×DBA/2 mice as compared to control CBA/J×BALB/c mice (see Example 19);

FIG. 16C provides a graph of sFlt-1 production in monocytes from CBA/J×DBA/2 mice as compared to monocytes from animals carrying a genetic deletion of TF (TF^{floxed/floxed} LysM-Cre mice), where the monocytes are treated with LPS, C5a, anti-TF antibodies, or combination of anti-TF antibodies and C5a;

FIG. 16D provides assessment of cell proliferation of trophoblasts treated with sFlt-1 and the effect of adding VEGF;

FIG. 16E provides assessment of superoxide production by (a) trophoblasts incubated with media only, (b) trophoblasts incubated with supernatants of monocytes exposed to C5a and s-Flt-1, and (c) trophoblasts incubated with supernatants from monocytes exposed to C5a and s-Flt-1 but in presence of VEGF;

FIG. 16F provides results of staining for TF expression on trophoblasts incubated with supernatants from monocytes or with s-Flt-1, and trophoblasts incubated with supernatants from monocytes treated with s-Flt-1 in presence of VEGF;

FIGS. 17A, 17B and 17C provide graphs of the effect of pravastatin (P) treatment on fetal resorption, fetal weight, and litter size of CBA/J×DBA/2 mice as compared to control CBA/J×BALB/c mice;

FIG. 17D provides photographs of placental tissue assessed for oxidative stress, TF expression, and fibrin deposition from placentas of CBA/J×DBA/2 mice treated with pravastatin and in untreated animals;

FIG. 17E provides graphs of serum NO levels in untreated CBA/J×BALB/c mice, untreated CBA/J×DBA/2 mice; and CBA/J×DBA/2 mice treated with pravastatin;

FIG. 17F provides a photomicrograph of blood flow in the placenta of CBA/J×DBA/2 mice treated with pravastatin, showing restored placental blood flow;

FIG. 17G provides a FACs analysis for TF-positive monocytes of untreated CBA/J×DBA/2 mice and CBA/J×DBA/2 mice treated with pravastatin; and

FIG. 17H provides photomicrographs of trophoblasts treated with sFlt-1, or trophoblasts treated with sFlt-1 and pravastatin, and evaluated for superoxide production or −+TF expression.

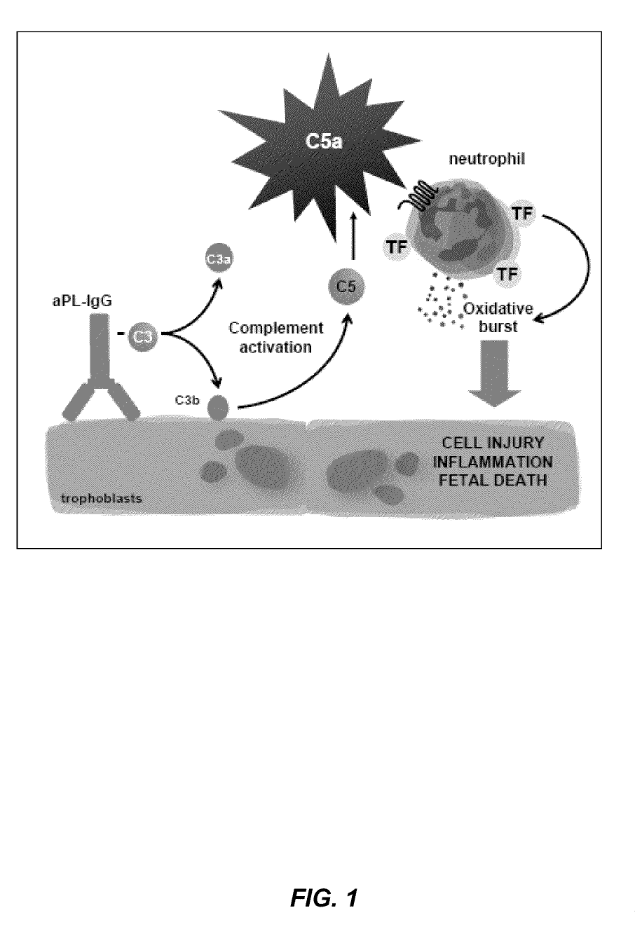

### DETAILED DESCRIPTION

The present disclosure concerns the use of compounds capable of inhibiting TF expression on neutrophils and/or monocytes, such as, among others, statins, as a therapeutic approach towards the prevention and/or reduction of pregnancy loss or miscarriage, especially in women who have suffered prior or recurrent miscarriages (whether of known or unknown origin) and/or who exhibit or suffer from APS, as well as methods of identifying patients at risk of miscarrying. The inventions described herein are based, in part, on the discovery that inhibition of TF expression prevents pregnancy loss in two different murine models of miscarriage.

As noted above, one form of pregnancy loss is associated with anti-phospholipid antibodies (aPL), also referred to as anti-phospholipid syndrome (APS). It has been postulated previously that aPL-induced pregnancy loss may involve two different cascade pathways: the inflammatory cascade and the coagulation cascade. Data presented in the Examples section demonstrate that inhibition of the coagulation cascade is not required for prevention of miscarriage. Rather, in both aPL-induced and non aPL-dependent murine models of miscarriage, antibodies specific for inhibition of TF-induced inflammation prevented miscarriage, whereas antibodies specific for TF-induced coagulation did not. A significant advantage of the use of statin TF-expression inhibitor compounds to prevent miscarriage is that they do not affect coagulation. Thus, they provide an effective means of preventing miscarriage that does not put the patient at risk of bleeding or excessive bleeding.

The second form of pregnancy loss described herein is based on a mouse model of recurrent spontaneous miscarriages, DBA/2-mated CBA/J mice, that shares features with human recurrent miscarriage, fetal growth restriction, and preeclampsia. Embryos derived from mating CBA/J females with DBA/2 males show an increased frequency of resorption when compared to control matings (CBA/J×BALB/c), and surviving fetuses show consistent and significant growth restriction. The embryos of these animals display other signs of placental abnormalities, such as increased oxidative stress, reduced angiogenesis, and reduced placental flow. In humans, recurrent pregnancy loss (RPL) is typically characterized as the occurrence three or more consecutive pregnancies that end in miscarriage of the fetus and affects 1% to 3% of couples. Similar to the mouse model, intrauterine growth restriction (IUGR) is another pregnancy complication that occurs in up to 10% of infants, and is a second leading cause of perinatal morbidity and mortality, following prematurity. Fetuses with IUGR are at high risk for poor short- and long-term outcomes. Moreover, IUGR, increased oxidative stress, abnormal angiogenesis and abnormal placental blood flow are observed in human preeclampsia. While preeclampsia is generally agreed to be associated with abnormal implantation and development of the placenta (first trimester), studies on the cause generally focus on examining events in the late second or third trimester when the maternal symptoms manifest. Since it is difficult to describe the progression of events with any confidence in human studies, mouse models have been useful as they can be used to study the onset and progression of preeclampsia.

In the aPL mediated pregnancy loss, increased presence of TF is found in the population of neutrophils of affected animals. It is shown herein that the DBA/2-mated CBA/J mice mouse model of non-aPL pregnancy loss is also characterized by an increase in presence of TF, similar to that observed in the aPL mediated pregnancy loss. In particular, increase in TF-positive cells is seen in the population of monocytes from affected animals. This increase in TF is associated with (a) increases in sFlt-1, the soluble form of the VEGF-1 receptor which has anti-angiogenic activity by sequestering VEGF; (b) increase in plasma thrombin-anti-thrombin III complex (TAT) levels throughout pregnancy; and (c) increases in soprostane 8-iso-prostaglandin F2a (STAT-8). The increase in plasma thrombin-anti-thrombin complex suggests that increased coagulation can play a role in pregnancy complications of the DBA/2-mated CBA/J mice while the STAT-8 can play a role in pregnancy loss through its potent vasoconstrictor and platelet activating properties as well as by inducing endothelial cell derangement and reducing trophoblast invasion. All of the foregoing features are known to be associated with preeclampsia. Importantly, increased TF expression is also observed in human placentas from growth restricted neonates of women with preeclampsia as compared to placentas from neonates with appropriate weight for gestational age.

TF expression in monocytes as an important indicator of non-aPL type of pregnancy loss is suggested by the findings that recombinant animals deleted for the TF gene show no increases in sFlt-1 release and that depletion of monocytes prevents pregnancy loss and IUGR in the mouse model. Indeed, inhibition of TF activity by use of antibodies to TF or by use of a statin, such as pravastatin, to inhibit TF expression on monocytes reduced sFlt-1 release from monocytes, reduced plasma levels of sFlt-1, prevented oxidative stress, decreased fibrin deposition, and restored placental blood flow. Importantly, administration of a TF expression inhibitor compound (i.e., an inhibitor of TF expression, for example a statin) or a TF activity inhibitor compound (i.e., an inhibitor of TF activity, for example an anti-TF antibody), prevented and/or reduced the amount of pregnancy loss, IUGR, and preeclampsia-like symptoms in these animals.

Given that increase in presence of TF, particularly in populations of monocytes and/or neutrophils, is associated with pregnancy loss or miscarriage, and that inhibition of TF expression results in the reduction of pregnancy loss, the methods can comprise administering to a patient who is either pregnant or planning to become pregnant a therapeutically effective amount of a TF-expression inhibitor or an TF-activity inhibitor to prevent and/or reduce the risk of pregnancy loss or miscarriage, particularly to prevent and/or reduce the risk of preeclampsia or IUGR.

In some embodiments, the methods can comprise administering to a patient who is either pregnant or planning to become pregnant a therapeutically effective amount of a TF expression inhibitor compound to prevent and/or reduce the risk of pregnancy loss or miscarriage, particularly to prevent and/or reduce the risk of preeclampsia of IUGR.

In some embodiments, the patient, either pregnant or planning to become pregnant manifests presence of anti-phospholipid antibodies associated with APS. For example, the patient can be one who had a previous miscarriage or suffered recurrent miscarriages and has been diagnosed with or is suspected of suffering from APS. In some instances, the patient can be one who has anti-phospholipid antibodies associated with APS and is pregnant for the first time or one who has anti-phospholipid antibodies associated with APS but has no previous miscarriages. In these instances, the patient can be administered an amount of a TF-expression inhibitor effective to prevent and/or reduce the likelihood pregnancy loss or miscarriage.

In some embodiments, the patient, either pregnant or planning to become pregnant, does not or has not manifested presence of anti-phospholipid antibodies associated with APS but has had a previous miscarriage or suffered recurrent miscarriages, or had preeclampsia in a previous pregnancy. In these instances, the patient can be administered an amount of a TF-expression inhibitor compound or a TF-activity inhibitor compound effective to prevent and/or reduce the likelihood of pregnancy loss or miscarriage, particularly to prevent and/or reduce the risk of preeclampsia or IUGR.

Other risk factors that can be considered include, among others, hypertension, obesity and diabetes. Patients with these conditions generally have increased risk of preeclampsia during pregnancy (see, e.g., Becker et al., 2008, J Obstet Gynaecol Can. 2008 30(12):1132-6; Leddy et al., 2008, J. Rev Obstet Gynecol. 2008 1(4):170-8; Edlow et al., 2008, Obstet Gynecol.; Voigt et. al., 2008, 212(6):201-5. Epub 2008; Yu et al., 2009, Diabetologia. 52(1):160-8. Epub 2008 Nov. 5; Yogev et al., 2008, Semin Fetal Neonatal Med.; and Kaaja, 2008, Ginecol. 60(5):421-9).

In light of the association of elevated TF levels with pregnancy loss and miscarriages, in some embodiments, increase in TF positive neutrophils, increase in TF-positive monocytes, increase in TF levels in blood, increase in TF levels in chorionic villus, or various combinations thereof, can be used as a basis for selecting a patient for therapeutic intervention. Use of TF levels as a criteria for therapy can be applicable to a patient who is pregnant or is planning to become pregnant for the first time, has not had a previous miscarriage nor suffered recurrent miscarriages, or has not manifested preeclampsia in a previous pregnancy; but has not manifested presence of anti-phospholipid antibodies associated with APS. In some embodiments, the use of elevated TF levels as a criteria for therapy can be applicable to a patient who is pregnant or planning to become pregnant and has had a previous miscarriage, suffered recurrent miscarriages, or manifested preeclampsia in a previous pregnancy but has not manifested presence of anti-phospholipid antibodies associated with APS or has not been previously diagnosed with APS.

Thus, in some embodiments, the method comprises administering to a patient who is pregnant or planning to become pregnant and has increased numbers of TF-positive neutrophils as compared to the numbers of TF positive neutrophils in normal pregnant women or a normal non-pregnant women, respectively, an amount of an TF expression inhibitor compound or TF-activity inhibitor compound effective to prevent and/or reduce the likelihood of pregnancy loss or miscarriage, particularly to prevent and/or reduce the likelihood of preeclampsia or IUGR.

In some embodiments, the methods comprise administering to a patient who is pregnant or planning to become pregnant and has increased numbers of TF-positive monocytes as compared to numbers of TF-positive monocytes in normal pregnant women or a normal non-pregnant women, respectively, an amount of a TF-expression inhibitor compound or TF-activity inhibitor compound effective to prevent and/or reduce the likelihood of pregnancy loss or miscarriage, particularly to prevent and/or reduce the likelihood of preeclampsia or IUGR.

In some embodiments, the methods comprise administering to a patient who is pregnant or planning to become pregnant and has increased levels of TF present in the blood as compared to levels of TF present in blood of normal pregnant women or normal non-pregnant women, respectively, an amount of a TF-expression inhibitor compound or TF-activity inhibitor compound effective to prevent and/or reduce the likelihood of pregnancy loss or miscarriage, particularly to prevent and/or reduce the likelihood of preeclampsia or IUGR.

In some embodiments, the methods comprise administering to a patient who is pregnant and has increased levels of TF present in the chorionic villus as compared to levels of TF present in chorionic villus of normal pregnant women an amount of a TF-expression inhibitor compound or TF-activity inhibitor compound effective to prevent and/or reduce the likelihood of pregnancy loss or miscarriage, particularly to prevent and/or reduce the likelihood of preeclampsia or IUGR.

In some embodiments, the methods comprise administering to a woman who is pregnant or planning to become pregnant and has various combinations of increased numbers of TF positive neutrophils, increased numbers of TF positive monocytes, increased levels of TF in blood, and increased levels of TF in chorionic villus as compared to those levels in normal pregnant woman or a normal non-pregnant women, respectively, an effective amount of a TF-expression inhibitor compound or TF-activity inhibitor compound to prevent and/or reduce the likelihood of pregnancy loss or miscarriage, particularly to prevent and/or reduce the likelihood of preeclampsia or IUGR.

As described further below, an effective amount of a TF expression inhibitor compound can be an amount that is effective in inhibiting or reducing the presence or expression of TF in populations of neutrophils, populations of monocytes, or in maternal blood in patients with elevated levels of TF. A therapeutically effective amount can be an amount that prevents or reduces the likelihood of pregnancy loss or miscarriage, particularly to prevent or reduce the likelihood of preeclampsia or IUGR.

The level of TF as an indicator for therapeutic intervention can be determined by one of skill in the art, for instance, by comparing the numbers of TF positive neutrophils, the number of TF-positive monocytes, the levels of blood TF or chorionic villus TF of pregnant patients affected by APS, RPL of non-aPL origin (e.g., unknown origin), or preeclampsia, to those found in subjects with normal pregnancies or, where appropriate, normal non-pregnant subjects.

Exemplary levels of TF expression in normal pregnant mice is shown herein to be about 6% of neutrophils staining positive for TF, while in normal pregnant mice is shown herein to be about 6% of monocytes staining positive for TF. By comparison, in the mouse model of RPL about 30% of monocytes stain positive for TF while in the mouse model of aPL induced APS about 23% of neutrophils stain positive for TF. Thus, the increase in numbers of TF-positive neutrophils and/or monocytes for therapeutic intervention can be an increase of about 20% or more, 30% or more, 50% or more, 100% or more, 200% or more, 300% or more of the number of TF positive neutrophils and/or monocytes found in mothers with normal pregnancy. In some embodiments, for a pregnant patient, consideration for therapeutic intervention can be based on a single determination of TF-positive neutrophil and/or monocyte in a sample, or based on multiple measurements made over a defined time period and assessing any rise in TF levels as a function of time. The interval between measurements can be about 1 week, about 2 weeks, about 3 weeks, about 4 weeks, or another time interval determined to be appropriate by those skilled in the art. In particular, subjects with elevated numbers of TF-positive neutrophils and/or monocytes but without any clinical symptoms of preeclampsia or IUGR can be considered for prophylactic treatment to prevent or reduce the likelihood of pregnancy loss, particularly to prevent and/or reduce the likelihood of preeclampsia or IUGR.

In some embodiments, increased levels of TF in maternal blood can be used as a basis for considering therapeutic intervention. Presence of TF can be measured by a variety of methods, such as, among others, use of antibodies to TF (e.g., TF antibody based ELISAs or radioimmune assay) and anticoagulation assays. An exemplary method of determining TF levels in maternal blood and baseline values in normal pregnancies are described in Erez et al., 2008, J Matern Fetal Neonatal Med. 21(12):855-69, incorporated herein by reference. TF levels in maternal blood normal pregnancies appear to have a median of about 291.5 pg/mL, with a range of 6.3-2662.2, while levels in pregnancies with preeclampsia show a median of about 1187 pg/mL, with a range of 69-11675. In some embodiments, subjects with blood levels of TF above the median but without clinical symptoms of preeclampsia can be considered for treatment with a TF-expression inhibitor compound, e.g., statin, as a prophylactic measure to prevent or reduce the likelihood of pregnancy loss or miscarriage, particularly to prevent or reduce the likelihood of preeclampsia or IUGR. Treatment can be considered based on a single measurement of blood TF or based on multiple measurements made over a period of time, such as an interval of about 1 week, about 2 weeks, about 3 weeks, or about 4 weeks.

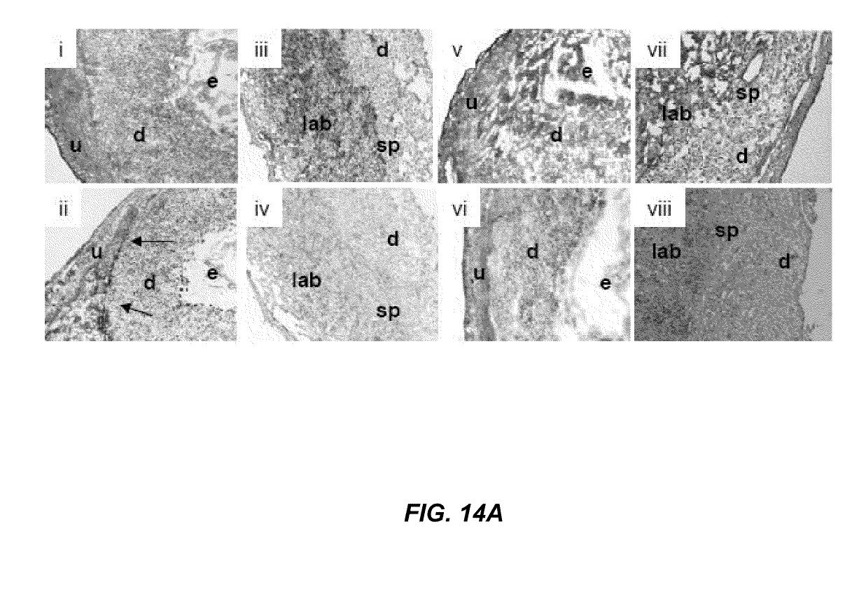

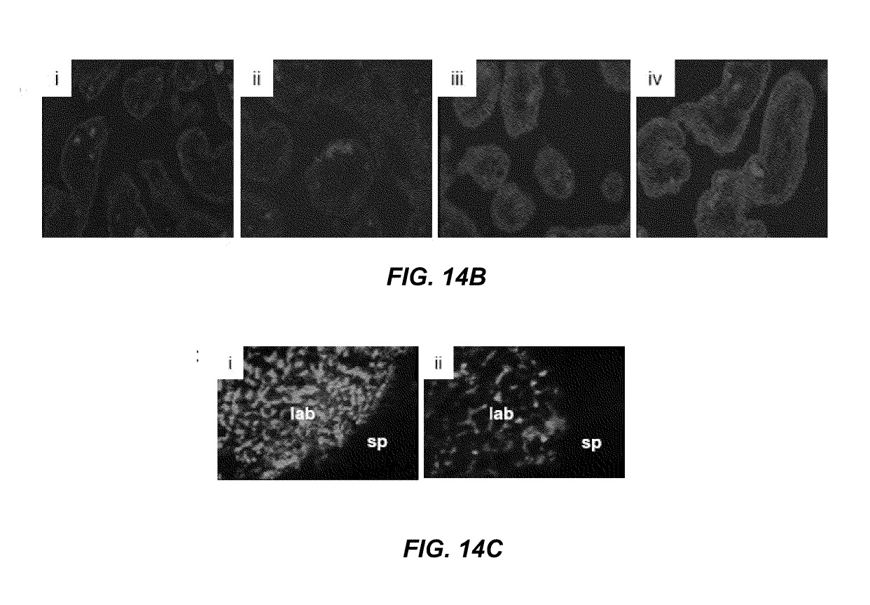

In some embodiments, increased levels of TF in the chorionic villus of a patient who has undergone a chorionic villus sampling (CVS) procedure can be used as a basis for considering therapeutic intervention (see, e.g., FIG. 14B). The presence of TF can be measured similarly to those techniques used for measuring blood TF or by in situ detection of TF in the placenta sample (see, e.g., FIG. 14B). Patients who show elevated levels of TF expression and/or increased numbers of TF-positive cells as compared to TF expression and/or numbers of TF-positive cells in normal pregnant patients can be selected for therapy.

As touched upon above, the levels of TF expressed or present on neutrophils, monocytes, maternal blood, or chorionic villus can be considered in combination with other criteria, such as past or present medical history (e.g., previous miscarriages, previous diagnosis of preeclampsia, obesity, diabetes, hypertension, etc.), growth of the fetus, level of blood flow to the fetus (e.g., as assessed by Doppler ultrasound), and/or other expression markers.

In some embodiments, the expression of TF can be used with one or more different markers, such as increased expression of sFlt-1 and/or increase in STAT8 activity. As described herein, increased expression of sFlt-1 is associated with increased expression of TF. In the absence of TF activity, sFlt-1 expression is reduced or not observed, indicating that TF is critical for s-Flt-1 expression. Use of and methods for detecting s-Flt-1 as a marker for preeclampsia is described in US application publication no. 20040126828 and US application publication no. 2004/0018201, incorporated herein by reference.

As used herein, a “TF expression inhibitor compound”, also described as a “TF inhibitory compound” refers to a compound or composition that is capable of reducing or inhibiting expression of TF on neutrophils and/or monocytes, such as by inhibiting transcription of a gene encoding TF, thereby blocking synthesis of TF mRNA; or inhibiting translation of mRNA encoding TF, thereby blocking synthesis of TF.

While the methods are exemplified by example with two exemplary statins (simvastatin and pravastatin), it is expected that any molecule that inhibits TF expression on neutrophils and/or monocytes will provide benefit in preventing and/or reducing pregnancy loss or miscarriage. Such molecules include, but are not limited to, antisense oligonucleotides, siRNAs, miRNAs and small molecules.

In a particular embodiment, the TF expression inhibitor compound is a statin. Statins are a well-known class of compounds that inhibit HMG-CoA reductase as a therapeutic approach towards treating high cholesterol. As mentioned in the Summary, statins have been shown to inhibit TF expression in various cell types (Pierangeli et al., 2005, J Thromb Haemost 3(5):1112-3; Kunieda, 2003, Thromb Res 110(4):227-34) and simvastatin has been shown to inhibit TF expression and activity in blood monocytes in patients with hepatic syndrome (Wei, 2007, Eur J Med Res 12(5):216-21). Indeed, data presented in the Examples section demonstrate that two exemplary statins, simvastatin and pravastatin, reduce pregnancy loss in mice in a murine model of APS, and that the exemplary statin pravastatin reduces pregnancy loss in a murine model of recurrent miscarriage of unknown origin (non aPL-induced).

The statins are a well-reorganized class of molecules, and either have a side chain that shares structural similarity to HMG-CoA (for example, mevastatin, lovastatin, simvastatin and pravastatin) or an HMG-CoA intermediate (for example, fluvastatin, atorvastatin, cerivastatin). All of these statins, as well as statins developed in the future, are expected to be useful in the methods described herein.

Statins have been categorized based on whether the statin is lipophilic or hydrophilic, and these different categories of statins can differ with respect to their tissue specificity and pharmacokinetics. Both of these categories of statins can be used for the methods herein. In some embodiments, the statin administered comprises a lipophilic statin, which include, among others, simvastatin, cerivastatin, atorvastatin, and lovastatin. In some embodiments, the statin administered comprises a hydrophilic statin, which including, among others, fluvastatin, rosuvastatin, and pravastatin. In some embodiments, combinations of statins, such as between or within the described categories, can be administered.

The statins can be administered in the form of the free acids or bases, or in the form of pharmaceutically-acceptable salts, for example, acid addition salts. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-tuluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. The free-bases and various pharmaceutically-acceptable salts of the statins listed above are well-known, and are available commercially.

In some embodiments, the TF expression inhibitor compound is an anti-tissue factor interfering RNA, also referred to as siRNA or RNAi. siRNAs are typically short 21 to 25 nucleotide double stranded RNAs or oligonucleotides capable of inhibiting or reducing expression of target genes. The siRNA can be chemically synthesized or produced by recombinant techniques and then introduced into cells directly or by some delivery system. Guidance on designing siRNAs are described in, among others, WO0175164 and U.S. Pat. No. 7,056,704. siRNA can be based on nucleobase polymers of purines and pyrimidines linked by sugar phosphate linkages, as found in nature, or nucleobase-polymers with modified nucleobases and non-natural internucleoside linkages. Modified backbones can include, among others, phosphorothioates, phosphotriesters, phosphoramidates, peptide nucleic acids (PNA), and morpholino based oligomers (e.g., U.S. Pat. No. 5,698,685 and US20060063150). Modified bases include, among others, xanthine, 7-methylaguanine, 3-deazaadenine, 5-urancil, 6-azouracil, etc. Exemplary siRNAs for mouse TF are described in Amarzauioui et al., 2006, Clin Cancer Res. 12(13):4055-61 while siRNAs for human TF are described in Holen et al., 2002, Nucleic Acids Res. 30(8): 1757-1766. In some embodiments, the siRNA can have the sequence and structure recited in Table 1.

TABLE 1

| | |
|---|---|
| siRNA Sequence | SEQ ID NO. |

| | |
|---|---|
| 5′-GCGCUUCAGGCACUACAAATT | SEQ ID NO: 1 |
| TTCGCGAAGUCCGUGAUGUUU | |
| 5′-GAAGCAGACGUACUUGGCATT | SEQ ID NO: 2 |
| TTCUUCGUCUGCAUGAACCGU | |
| 5′-CCCGUCAAUCAAGUCUACATT | SEQ ID NO: 3 |
| TTGGGCAGUUAGUUCAGAUGU | |

Other interfering RNAs and methods of screening for other siRNAs targeting TF expression are described in the art, for example US20050096289, incorporated herein by reference.

In some embodiments, the TF expression inhibitor compound comprises an antisense oligonucleotide for TF that is capable of inhibiting or reducing expression of TF on neutrophils and/or monocytes. The antisense oligonucleotide can be directed against (i.e., complementary to), among others, the RNA sequence of the 5′ leader region, the intron splicing sites, exon splicing sites, the intron-exon boundaries, the 3′ untranslated region, and internal regions of exons required for splicing reactions. As with the siRNAs, antisense oligonucleotides can be based on oligonucleotides with sugar-phosphate backbones and naturally occurring nucleobases, or based on modified backbones and modified nucleobases. Modified backbones can include, among others, phosphorothioates, phosphotriesters, phosphoramidates, peptide nucleic acids (PNA), and morpholino based oligomers. Modified bases include, among others, 5-methylcytosine (5-me-C), 5-hydroxymethyl cytosine, 2-aminoadenine, 6-methyl adenine or guanine, 2-thiouracil, 2-thiothymine, 2-thiocytosine, 5-halouracil, 5-propynyl uracil, 6-azo uracil, 5-uracil (pseudouracil), 4-thiouracil; 7-methylguanine, 7-methyladenine, 8-azaguanine, 8-azaadenine, 7-deazaguanine, 7-deazaadenine, 3-deazaguanine, and 3-deazaadenine, etc. Exemplary antisense oligonucleotides affecting tissue factor expression can be selected from the following:

TABLE 2

| | | |
|---|---|---|
| Target Site | | |
| (nt position) | TF Antisense Sequence | SEQ ID NO. |

| | | |
|---|---|---|
| 355 | actggtagacatggagaccc | SEQ ID NO:4 |
| 342 | gatctcgccgccaactggta | SEQ ID NO: 5 |
| 1695 | ttggagtgggaacccaaacc | SEQ ID NO: 6 |
| 319 | tacactgttcaaataagcac | SEQ ID NO: 7 |
| 326 | ttcaaataagcactaagtca | SEQ ID NO: 8 |
| 5856 | aaaagcaaatgcttttacac | SEQ ID NO: 9 |
| 5886 | gagtgtgacctcaccgacga | SEQ ID NO: 10 |
| 5891 | tgacctcaccgacgagattg | SEQ ID NO: 11 |
| 5896 | tcaccgacgagattgtgaag | SEQ ID NO: 12 |
| 5901 | gacgagattgtgaaggatgt | SEQ ID NO: 14 |
| 5925 | cagacgtacttggcacgggt | SEQ ID NO: 15 |
| 5943 | gtcttctcctacccggcagg | SEQ ID NO: 16 |
| 518 | tcacaccttacctggagaca | SEQ ID NO: 17 |
| 526 | tacctggagacaaacctcgg | SEQ ID NO: 18 |
| 8746 | gccaacaattcagagttttg | SEQ ID NO: 19 |
| 8804 | gaacggactttagtcagaag | SEQ ID NO: 20 |
| 8818 | cagaaggaacaacactttcc | SEQ ID NO: 21 |
| 8839 | aagcctccgggatgtttttg | SEQ ID NO: 22 |
| 8855 | tttggcaaggacttaattta | SEQ ID NO: 23 |
| 9535 | aaacactaatgagtttttga | SEQ ID NO: 24 |
| 9617 | gttaaccggaagagtacaga | SEQ ID NO: 25 |
| 9646 | agagtgtatgggccaggaga | SEQ ID NO: 26 |
| 9660 | aggagaaaggggaattcaga | SEQ ID NO: 27 |
| 11415 | tcattggagctgtggtattt | SEQ ID NO: 28 |
| 11420 | ggagctgtggtatttgtggt | SEQ ID NO: 29 |
| 11465 | atatctctacacaagtgtag | SEQ ID NO: 30 |
| 11470 | tctacacaagtgtagaaagg | SEQ ID NO: 31 |
| 11502 | agagctggaaggagaactcc | SEQ ID NO: 32 |
| 11542 | gaagcactgttggagctact | SEQ ID NO: 33 |
| 11585 | ccgagaacttttaagaggat | SEQ ID NO: 34 |
| 11601 | ggatagaatacatggaaacg | SEQ ID NO: 35 |
| 11625 | tgagtatttcggagcatgaa | SEQ ID NO: 36 |
| 11691 | agcattctggttttgacatc | SEQ ID NO: 37 |
| 11707 | catcagcattagtcactttg | SEQ ID NO: 38 |
| 11768 | ttttaacaccatggcacctt | SEQ ID NO: 39 |
| 11805 | tagattatatatteegeact | SEQ ID NO: 40 |
| 11837 | aggtcgtccaagcaaaaaca | SEQ ID NO: 41 |
| 11854 | acaaatgggaaaatgtctta | SEQ ID NO: 42 |
| 11875 | aaaatcctgggtggactttt | SEQ ID NO: 43 |
| 11880 | cctgggtggacttttgaaaa | SEQ ID NO: 44 |
| 11886 | tggacttttgaaaagctttt | SEQ ID NO: 45 |
| 12226 | cttcaatccatgtaggaaag | SEQ ID NO: 46 |
| 12233 | ccatgtaggaaagJaaaatg | SEQ ID NO: 47 |
| 12265 | gtgcatttctaggacttttc | SEQ ID NO: 48 |
| 12274 | taggacttttctaacatatg | SEQ ID NO: 49 |
| 12366 | tgtattaatgtgttaagtgc | SEQ ID NO: 50 |
| 12421 | gctttacaatctgcacttta | SEQ ID NO: 51 |
| 12493 | tactatacaaactacagagt | SEQ ID NO: 52 |
| 12516 | tgatttaaggtacttaaagc | SEQ ID NO: 53 |
| 12526 | tacttaaagcttctatggtt | SEQ ID NO: 54 |
| 12539 | tatggttgacattgtatata | SEQ ID NO: 55 |
| 12650 | tactttaaataaaggtgact | SEQ ID NO: 56 |
| 6027 | tacctggagagtaagtggct | SEQ ID NO: 57 |
| 6848 | agaacagtttctcaaggtag | SEQ ID NO: 58 |
| 8723 | tttgtttcagcaaacctcgg | SEQ ID NO: 59 |
| 9354 | agtgatcaagggaaactgat | SEQ ID NO: 60 |

Other exemplary antisense TF oligonucleotides and methods of generating such anti-sense oligonucleotides are described in US20040102402 and Nakamura et al., 2001, Transplant Proc. 33:3707-3708, incorporated herein by reference. Antisense oligonucleotides based on morpholino oligomers can be particularly effective in reducing or inhibiting expression of target genes (e.g., U.S. Pat. No. 5,698,685 and US20060063150). It is further to be understood that additional siRNAs and antisense oligonucleotides may be developed based on the mRNA sequence for human TF available at Genbank Accession no. M16553) by screening for siRNAs and antisense oligonucleotide capable of inhibiting expression of TF in monocytes and/or neutrophils.

In some embodiments, the patient can be administered an effective amount of a TF activity inhibitor compound. As used herein, a “TF activity inhibitor compound” refers to a compound or composition that inhibits the activity of TF, such as an antibody against TF, that reduces or inhibits TF signaling activity, particularly TF activity that inhibits TF-VIIa signaling through PAR2. In particular, antibodies against TF can be used to prevent and/or reduce the likelihood of aPL-mediated or non-aPL mediated, particularly non-aPL mediated pregnancy loss or miscarriage. An exemplary antibody of this type is 10H10, as described in Versteeg et al., 2008, Blood 111(1):190-9, Epub 2007 Sep. 27, incorporated herein by reference.

The TF expression inhibitor compound or salt, or the TF activity inhibitor compound can be administered in the form of the compound or salt per se, or alternatively, in the form of a composition formulated for a specific route or mode of administration, including, by way of example and not limitation, intravenous, intramuscular, subcutaneous, sublingual, buccal, nasal or oral administration. Ingredients and methods of formulating compounds into dosage forms suitable for use in these various modes of administration are well-known. Immediate and sustained release dosage forms of statins and/or their salts useful for oral administration are available commercially. For example, an oral dosage form of atorvastatin calcium is available from Pfizer under the trade name LIPITOR®, an oral dosage form of simvastatin is available from Merck under the tradename ZOCOR®, an oral dosage form of lovastatin is available commercially from several generic sources, including Alphama, Inc., Eon Labs and Mylen Laboratories, Inc., and an oral dosage form of pravastatin sodium is available from Bristol-Myers Squibb under the tradename PRAVACHOL®. All of these commercially available oral dosage forms can be used in the methods described herein.

The TF expression inhibitor compound or composition or TF activity inhibitor compound or composition will generally be administered in an amount effective to provide benefit; that is, in an amount effective to prevent or reduce loss of pregnancy or miscarriage. The compound or composition can be administered after the onset of pregnancy, or prior to conception. The therapy can be discontinued after a specified time period, such as after a period associated with high risk of pregnancy loss, for example, the first trimester, or it can be continued throughout the duration of pregnancy until delivery.

For a planned pregnancy, therapy can be initiated approximately one-month in advance of expected conception. For an unplanned pregnancy, therapy can be initiated at any time after pregnancy is appreciated. In some embodiments, it is preferable to begin therapy as soon as possible after a positive pregnancy test.

Animal data presented herein demonstrate that two exemplary statin TF expression inhibitor compounds, simvastatin and pravastatin, also prevent intrauterine growth restriction (IUGR). IUGR occurs in up to 10% of infants born in the U.S., and fetuses with IUGR are at high risk for poor short- and long-term outcomes. Owing to the demonstrated ability of the TF expression inhibitor compounds to prevent IUGR, continuing therapy throughout pregnancy up to childbirth is expected to reduce IUGR, and provide additional benefit. Accordingly, in some embodiments, the TF expression inhibitor compound therapy is applied either from before conception or just thereafter, and continued until birth of the child.

As described herein, the patient can be a normal, healthy woman who is pregnant or planning to become pregnant for the first time, or who has experienced one or more prior pregnancies. The patient can be a woman who is pregnant and shows elevated TF on monocytes, elevated TF on neutrophils, elevated TF in blood, elevated TF in chorionic villus, or various combinations thereof. TF expression can be measured at the level of RNA or protein. Expression of RNA encoding TF is readily determined by various techniques known in the art, such as by in situ hybridization, polymerase chain reaction, and membrane hybridization techniques. Expression of TF protein is readily determined by use of antibodies to TF and detecting the antibody-TF complex (e.g., ELISA, radioimmune assay, immunohistochemical assay, etc.).

While not intending to be bound by any theory of operation, it is believed that the therapy will be particularly suited to reducing the likelihood of, or preventing pregnancy loss or miscarriage, particularly preventing and/or reducing the likelihood of preeclampsia, in women who have experienced recurrent miscarriages (of either known or unknown origin) and/or who exhibit or suffer from APS (and therefore experience aPL-induced miscarriage).

Initial dosages expected to be effective can be estimated from in vitro or in vivo animal data, such as the animal models described in the Examples section. For simvastatin, dosages of 80 mg/day (administered orally) were found to blunt expression of endotoxin-induced monocyte TF expression. This dosage is expected to be useful in the methods described herein. For statins generally, the dosages typically used to lower cholesterol are expected to be effective in the methods described herein. Guidance regarding such dosages can be found in The Physician's Desk Reference, current edition, and/or in the product insert and/or labeling for the specific statin being used.

Patients can be monitored during therapy for presence of TF positive neutrophils, TF positive monocytes, blood levels of TF, or combinations thereof and the dosage adjusted accordingly. For example, a patient can be administered between about 10 mg/day, to about 20, 25, 30, 35, 40, 45, 50, 55, 60, 65, 70, 75, 80, 85, 90, 95 or even 100 mg/day statin and the level of neutrophil-expressed and/or monocyte-expressed TF assessed and the dosage adjusted accordingly. Assays suitable for monitoring TF expression on neutrophils and monocytes are described in the Examples and in Redecha et al., 2007, Blood 110(7):2423-2431, the disclosure of which is incorporated by reference. Generally, dosage should be adjusted until the presence of TF-positive cells in the population of neutrophils and/or population of monocytes, or the level in maternal blood is reduced to those present in normal pregnant patients, or, where appropriate, to levels present in normal non-pregnant patients.

Various representation embodiments having been described, the invention described herein are further illustrated with reference to the Examples and figures that follow.

### EXAMPLES

### Example 1

### Simvastatin Prevents Fetal Loss in a Murine Model of APS

This Example demonstrates that mice treated with aPL antibodies have increased rates of fetal resorption, and that treatment with simvastatin prevents fetal loss.

Protocol. Pregnant mice (n=7-10 per group) were treated with simvastatin (20 μg 18 hours prior to aPL or NHIgG injections on days 8 and 12). Simvastatin (Sigma Chemical) was prepared as a 4 mg/ml stock. Briefly 4 mg of simvastatin was dissolved in 100 μl of ethanol and 150 μl of 0.1N NaOH and incubated at 50° C. for 2 h, then the pH was adjusted to 7, and the total volume was corrected to 1 ml. The stock solution was diluted to the appropriate concentration in sterile PBS.

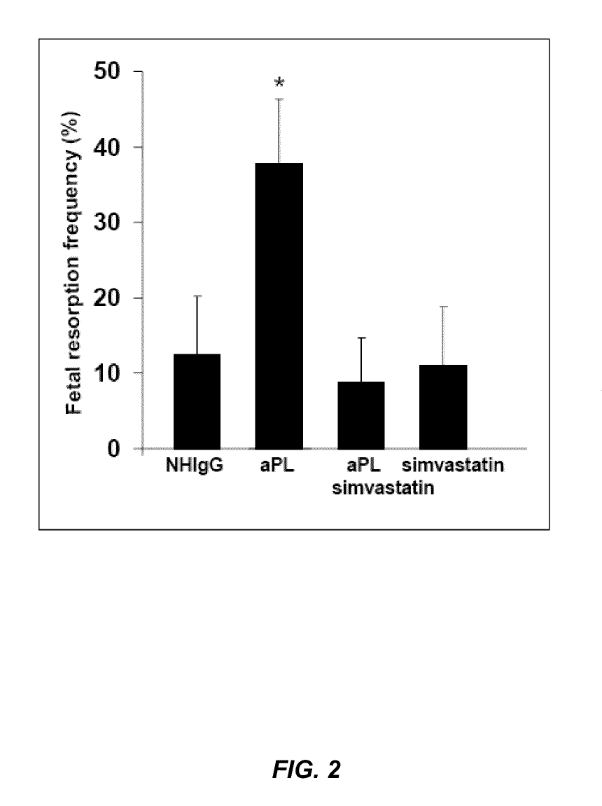

Results. The results are shown in FIG. 2. For each group, the resorption rate was calculated as number of resorptions per total number of formed fetuses and resorptions. Statistically significant differences were observed between aPL-treated mice and mice that received control IgG (NHIgG). aPL vs NHIgG (p<0.005). Statistically significant difference was also observed between aPL and aPL+simvastatin (p<0.005).

### Example 2

### Simvastatin Reduces Pregnancy Loss in a Murine Model of APS

This example demonstrates that simvastatin rescues pregnancies in mice treated with aPL antibodies.

Protocol. Pregnant C57BL/6 mice were treated with IgG (10 mg i.p.) from a healthy non-autoimmune individual (NH-IgG), or different patients with APS (aPL-IgG) on days 8 and 12 of pregnancy. Mice were sacrificed on day 15 of pregnancy, uteri were dissected, fetuses were weighed, and frequency of fetal resorption calculated (number of resorptions/number of fetuses+number of resorptions).

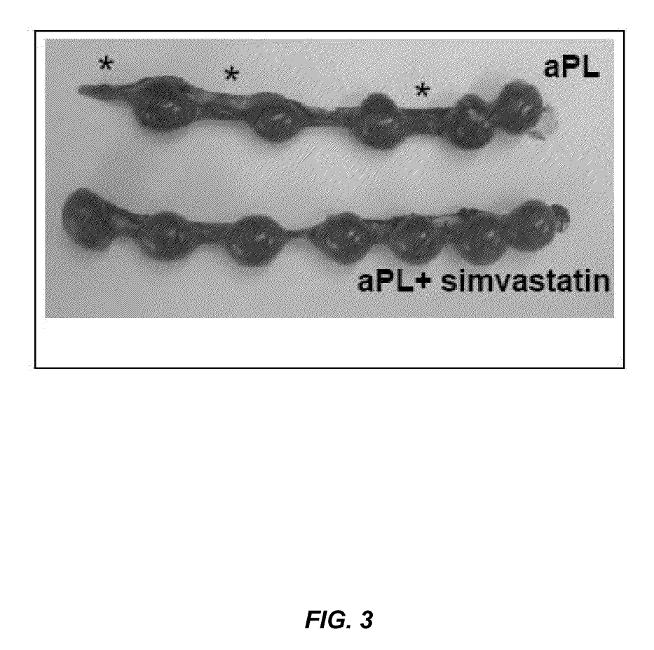

Results. The results are shown in FIG. 3. On the top panel is a uterus from a mouse treated with aPL. There are multiple fetal resorptions (miscarriages)-indicated by the asterisks. On the bottom there is a uterus from a mouse treated with aPL+simvastatin, there are 7 viable fetuses and no fetal resorptions comparable to NH-IgG treated mice.

### Example 3

### Simvastatin Inhibits aPL-Induced TF Expression, ROS Production and Phagocytosis in Neutrophils

Protocol. To study the presence of TF on peripheral blood neutrophils, two-color FACS analysis was performed. For this purpose heparinized whole mouse blood was stained with FITC-labeled anti-mouse Ly-6G (Gr-1) for FL1 (BD Biosciences Pharmingen, CA) to identify neutrophils and biotinylated rat anti-mTF antibody 1H1 and streptavidin-PerCP (BD Biosciences Pharmingen, CA) as the FL3 fluorochrome. Red cells were lyzed with ACK buffer. Preparations were then incubated with SA-PerCP and analyzed by FACS using a FACscan (BD Biosciences, CA).

Respiratory burst activity in neutrophils. Intracellular reactive oxygen (ROS) production was assessed with dihydrorhodamine 123 (DHR, Sigma Chemical, St Louis, Mo.) by flow cytometry. This primarily non-fluorescent dye becomes fluorescent upon oxidation to rhodamine by ROS produced during the respiratory burst. DHR (10 μmol/L) was added to heparanized whole blood, and this mixture was incubated at 37° C. for 45 min. Red cells were lysed as described above and reactive-oxygen production was analyzed by absorbance in FL1.

Phagocytosis assay. Heparanized whole blood was incubated (1 h, 37° C., 5% CO₂) with 1 μm of fluorescent yellow-green latex beads (Sigma-Aldrich) (ratio phagocyte/target 1:25) that had been preopsonized with normal mouse serum for 1 h. Internalization of beads was determined by flow cytometry using a FACSCalibur (BD Biosciences) flow cytometer.

Results. The results are tabulated in Table 1, below:

TABLE 1

| Treatment | TF | ROS | Phagocytosis |
|---|---|---|---|
| NH IgG | 6.4 ± 3.3 | 10.5 ± 6 | 20.6 ± 2.6 |
| aPL | 22.8 ± 5.9 | 34.4 ± 15.2 | 48.2 ± 15.9 |
| aPL + Simvastatin | 10 ± 5.7 | 10.4 ± 5.4 | 16.2 ± 2.9 |
| Simvastatin | 6.6 ± 2.2 | 10.4 ± 4.5 | 18.7 ± 1.0 |

In Table 1, the numbers are expressed as % of positive cells measured by flow cytometry analysis. Neutrophils from aPL-treated mice showed increased TF expression on neutrophils (Increased number of TF positive cells). TF increases neutrophil activity as shown by ROS production and phagocytosis. Simvastatin prevents TF-dependent neutrophil activation.

### Example 4

### Simvastatin Inhibits aPL Antibody-Induced Increase in PAR-2 Expression

Protocol. TF expression on mouse neutrophils. To study the presence of PAR-2 on peripheral blood neutrophils, two-color FACS analysis was performed. For this purpose heparinized whole mouse blood was stained with FITC-labeled anti-mouse Ly-6G (Gr-1) for FL1 (BD Biosciences Pharmingen, CA) to identify neutrophils and biotinylated anti-PAR-2 antibody (Santa Cruz Biotechnology, Inc, Santa Cruz, Calif.) and streptavidin-PerCP (BD Biosciences Pharmingen, CA) as the FL3 fluorochrome. Red cells were lyzed with ACK buffer. Preparations were then incubated with SA-PerCP and analyzed by FACS using a FACscan (BD Biosciences, CA).

Results. The results (expressed as number of PAR-2 positive cells) are tabulated in Table 2, below:

TABLE 2

| Treatment | PAR-2 |
|---|---|
| NHIgG | 6.0 ± 2.0 |
| aPL | 12.3 ± 6.5 |
| aPL + simvastatin | 7.4 ± 3.9 |
| simvastatin | 6.9 ± 2.3 |

TF complexes and other coagulation proteases may act as cell signaling molecules via protease-activated receptors cleavage, subsequently increasing inflammatory responses. Activation of PAR-s might thus exacerbate inflammation contributing to placental damage in aPL-induced pregnancy loss. A significant increase in PAR-2 expression was found on the cell surface of neutrophils from aPL-treated mice compared with control mice that received NHIgG. Simvastatin inhibits aPL-induced PAR-2 increase. By inhibiting PAR-2 increase simvastatin prevents inflammation and fetal death.

### Example 5

### Simvastatin Blocks aPL Antibody-Induced Increase in TF and PAR-2 Synthesis

Protocol. TF and PAR-2 mRNA expression analysis. For real-time PCR, total RNA from kidney tissue and was extracted using a RNeasy Mini kit (Qiagen), and 1 μg of total RNA was reverse transcribed using a First Strand cDNA Synthesis kit (Fermentas). Relative quantification of gene expression was performed by real-time PCR using iQ SYBR-Green Supermix on the iCycler iQ thermal cycler (Bio-Rad Laboratories) following the manufacturer's protocols. Primer sequences were as follows: mouse GAPDH, sense primer 5′-ttcaccaccatggagaaggc-3′ (SEQ ID NO:61), antisense primer 5′-ggcatggactgtggtcatga-3′ (SEQ ID NO:62); mouse TF, sense primer 5′-agctactgcttttttgtacaagctatg-3′ (SEQ ID NO:63), antisense primer 5′-tgtaccgtttcgttcgtcctaa-3′ (SEQ ID NO:64); mouse protease-activated receptor 2 (PAR-2), sense primer 5′-tggccattggagtcttcctgtt-3′ (SEQ ID NO:65), antisense primer 5′-tagccctctgccttttcttctc-3′ (SEQ ID NO:66). Relative expression was normalized for levels of GAPDH. The generation of only the correct size amplification products was confirmed using agarose gel electrophoresis.

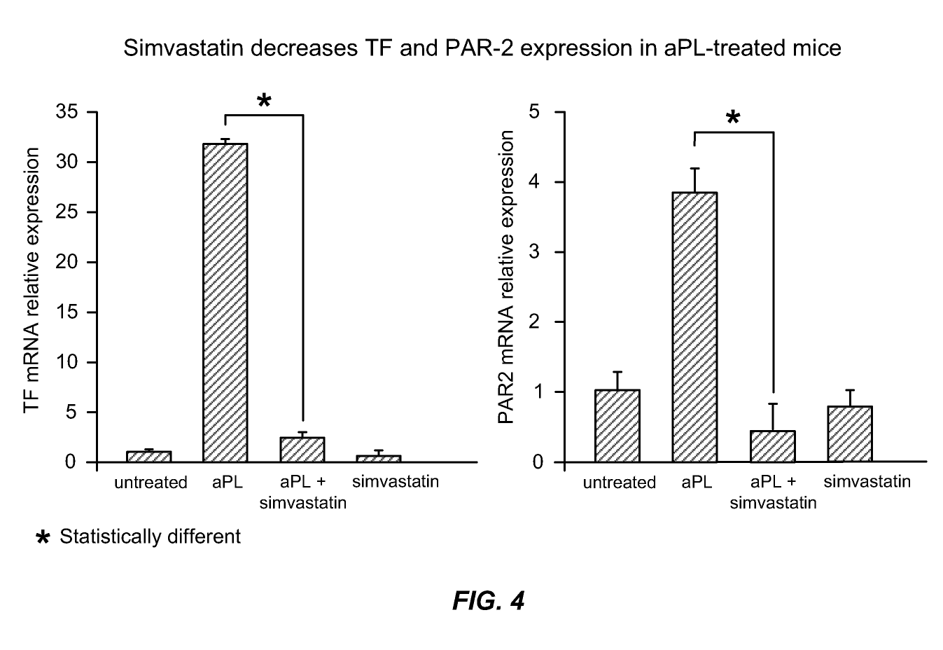

Results. The results are shown in FIG. 4. Real time PCR experiments show increased number of copies of TF and PAR-2 mRNA in neutrophils from aPL-treated mice. Simvastatin prevented this synthesis increase (*). By blocking synthesis and expression of TF and PAR-2 induced by aPL, simvastatin prevents neutrophil activation, placental damage and fetal death.

### Example 6

### aPL Antibody-Induces Increases in Free-Radical-Medicated Lipid Peroxidation in Decidual Tissue is Ameliorated by Simvastatin Treatment

Protocol. Assessment of superoxide production in decidual tissue by dihydroethidium fluorescence. In situ superoxide (O₂⁻) levels were assessed using the fluorescent probe dihydroethidium (DHE). Deciduas from day 8 of pregnancy were frozen in O.C.T. compound, cut into 10 μm sections and incubated with DHE (10 μmol/l) for 30 min at 37° C. Subsequently, the sections were washed and fluorescence images were obtained (λEx: 520, Em: 605 nm). To exclude an influence of the embedding procedure on fluorescence, samples from all treatment groups were embedded in the same block and analyzed simultaneously.

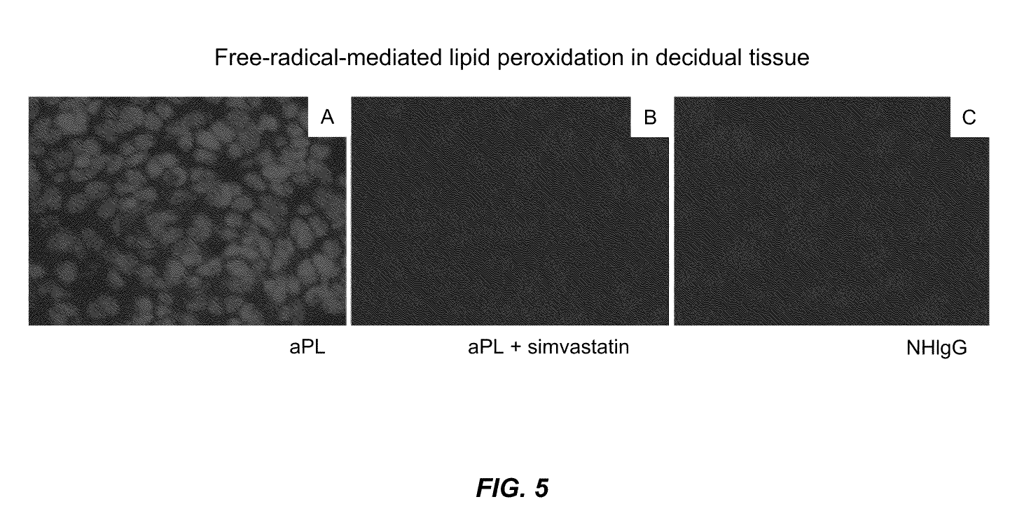

Results. The results are shown in FIG. 5. Superoxide production was measured in deciduas by dihydroethidium staining (DHE). Increased oxidative stress observed in deciduas from aPL-treated mice (A) is not observed when mice received simvastatin treatment (B). Free-radical-mediated lipid peroxidation in deciduas from aPL+simvastatin mice is comparable to deciduas from NHIgG-treated mice (C).

### Example 7

### Pravastatin Prevents Pregnancy Loss in a Murine Model of APS

This example demonstrates that another exemplary statin, pravastatin, also prevents aPL-induced pregnancy loss in a murine model of APS.

Protocol. Pregnant mice were treated with pravastatin (0.2 μg/g bw=5 μg/mouse, i.p 18 hours prior to aPL or NHIgG injections on days 8 and 12). Pravastatin (Sigma Chemical, St Louis, Mo.) was dissolved in EtOH at a concentration of 10 mg/mL and diluted with NaCl 0.9% in a ratio of 1:1000 to yield a final concentration of 10 μg pravastatin per 1 mL carrier. The NaCl 0.9%-based carrier solution for the placebo group was prepared accordingly to include EtOH only (i.e., without pravastatin) at a concentration of 1:1000.

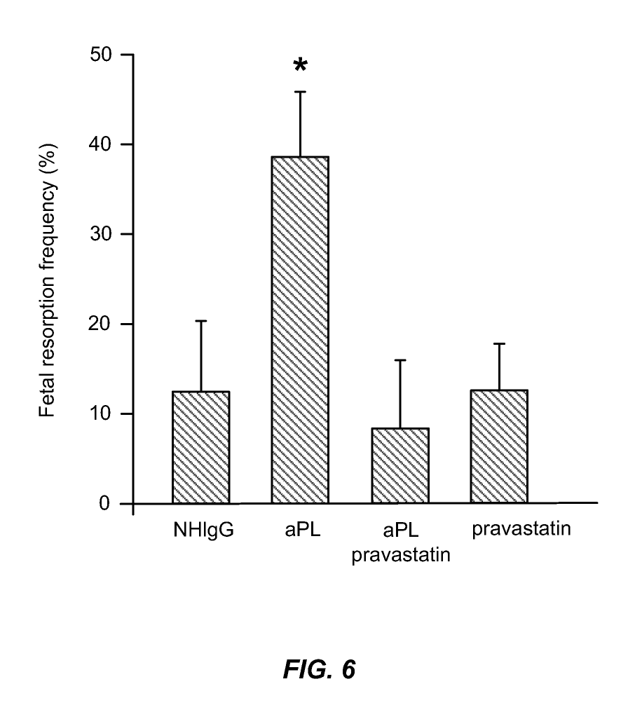

Results. The results are illustrated in FIG. 6. The resorption rate was calculated as number of resorptions per total number of formed fetuses and resorptions. Statistically significant differences were observed between aPL-treated mice and mice that received control IgG (NHIgG). aPL vs NHIgG (p<0.005). Statistically significant difference was also observed between aPL and aPL+pravastatin (p<0.05).

### Example 8

### Pravastatin Prevents Pregnancy Loss in a Murine Model of Non-aPL-Dependent Recurrent Miscarriage

This example demonstrates that the exemplary statin pravastatin is effective in preventing miscarriage in a murine model of recurrent miscarriage that is not aPL-dependent.

Protocol. DBA/2-mated female CBA/J mice (CBA/J×DBA/2) are a well-studied model of immunologically mediated pregnancy loss that shares features with human recurrent miscarriage (Clark, D A et al., 1998, J. Immunol. 160:545-549; Blois, M. et al., 2005, J. Immunol. 174:1820-1829). Embryonic lethality in DBA/2-mated female CBA/J mice is believed to represent rejection of the semiallogeneic placenta by maternal-derived activated effectors. 8-10-wk-old virgin female CBA/J mice were mated with 8-14-wk-old CBA/J, BALB/c, or DBA/2 males. Females were inspected daily for vaginal plugs, and presence of a vaginal plug was designated as day 0 of pregnancy. A group of DBA/2- and BALB/c mated CBA/J females were treated with pravastatin (0.2 μg/g bw=5 μg/mouse, i.p) on days 5, 6 and 7 of pregnancy. Previous studies from our laboratory show that fetal death occurs during this window time (Girardi et al., 2006, J Exp Med. 203(9):2165-75). Pregnant females were euthanized on day 15 and fetal resorption frequency was calculated.

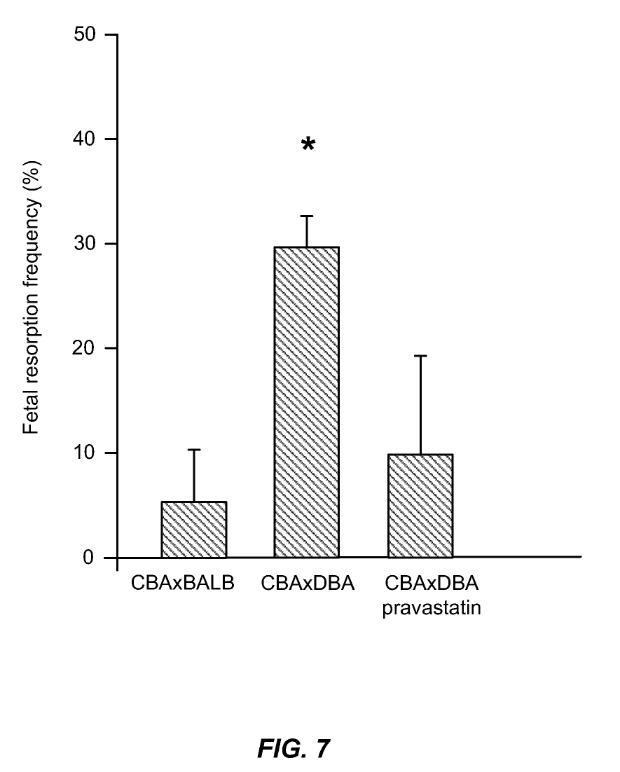

Results. Embryos derived from mating CBA/J females with DBA/2 males showed an increased frequency of resorption (30.1±2.8%), more than five times greater than that seen within CBA/J×BALB/c combination: 5.5±3.0%; P<0.01 (FIG. 7). FIG. 7 shows that pravastatin prevents fetal death in CBA/J×DBA/2 matings. Administration of pravastatin prevented fetal resorption in CBA/J×DBA/2 matings but has no effects on the outcomes of CBA/J×BALB/c matings (data not shown).

### Example 9

### Simvastatin and Pravastatin Prevents IUGR in a Murine Model of APS

This example demonstrates that two exemplary statins, simvastatin and pravastatin, prevent intrauterine growth restriction (IUGR) in a murine model of APS.

Increased fetal resorption is observed in mice treated with aPL and in CBA/J females mated with DBA/2 males. Resorption is not universal, however, and surviving fetuses show consistent and significant growth restriction (IUGR). Intrauterine growth restriction (IUGR) occurs in up to 10% of infants born in the United States (Resnik R et al, 2003, In Maternal-Fetal Medicine Principles and Practice. J. D. Iams, editor. Philadelphia, Pa.: Saunders. 495-512.) and fetal growth restriction is the second leading cause of perinatal morbidity and mortality, followed only by prematurity (Manning, F. A. 1995, In Fetal Medicine: Principles and Practice. Norwalk, Conn.: Appleton and Lange. 307). Fetuses with IUGR are at high risk for poor short- and long-term outcome. While the insult to the fetus occurs in utero, the deleterious influence of intrauterine growth restriction contributes to long-term developmental delay, and a sub-optimal intrauterine environment is a risk factor for development of coronary artery disease, diabetes, hypertension, hyperlipidemia and immune dysfunction in adult life (Hoet, J. J. et al., 1999, J Physiol 514 (Pt 3):617-627; Forsen et al., 1999, BMJ 319:1403-1407; Godfrey, K. M. et al., 2000, Am J Clin Nutr 71:1344 S-1352S; Veening, M. A. et al., 2002, J Clin Endocrinol Metab 4657-4661). Decreased intrauterine growth may possibly have a negative effect on brain growth and mental developmental potential. Children with a history of IUGR have been found to demonstrate attention and performance deficits.

Protocol. Pregnant mice were treated with pravastatin (0.2 μg/g bw=5 μg/mouse, i.p) or simvastatin (1 μg/g bw=0.8 μg/mouse, i.p) 18 hours prior to aPL or NHIgG injections on days 8 and 12 of pregnancy. Simvastatin (Sigma Chemical, St Louis, Mo.) was prepared as a 4 mg/ml stock. Briefly 4 mg of simvastatin was dissolved in 100 μl of ethanol and 150 μl of 0.1N NaOH and incubated at 50° C. for 2 h, then the pH was adjusted to 7, and the total volume was corrected to 1 ml. The stock solution was diluted to the appropriate concentration in sterile PBS. Pravastatin (Sigma Chemical, St Louis, Mo.) was dissolved in ethanol at a concentration of 10 mg/mL and diluted with NaCl 0.9% in a ratio of 1:1000 to yield a final concentration of 10 μg pravastatin per 1 mL carrier. The NaCl 0.9%-based carrier solution for the placebo group was prepared accordingly to include EtOH only (i.e., without pravastatin) at a concentration of 1:1000. On day 15 of pregnancy mice were euthanized and fetal weights were measured.

Results. The results of the simvastatin treatment group are shown in FIG. 8, the pravastatin treatment group in FIG. 9. Referring to FIG. 8, treatment with simvastatin prevented aPL-induced IUGR. aPL induced a 35% diminution in fetal weight. Fetal weights in mice treated with aPL+simvastatin were comparable to control mice (treated with NHIgG).

Referring to FIG. 9, pravastatin also prevented IUGR. No diminution in fetal weight was observed in mice treated with aPL+pravastatin when compared to mice treated with aPL alone.

### Example 10

### Pravastatin Prevents IUGR in a Murine Model of Non-aPL-Dependent Recurrent Miscarriage

This example demonstrates that the exemplary statin pravastatin also prevents IUGR in a murine model of recurrent miscarriage that is not aPL-dependent.

Protocol. 8-10-wk-old virgin female CBA/J mice were mated with 8-14-wk-old CBA/J, BALB/c, or DBA/2 males. Females were inspected daily for vaginal plugs, and presence of a vaginal plug was designated as day 0 of pregnancy. Pregnant mice were treated with pravastatin (0.2 μg/g bw=5 μg/mouse, i.p 18 hours prior to aPL or NHIgG injections on days 8 and 12). Pravastatin (Sigma Chemical, St Louis, Mo.) was dissolved in EtOH at a concentration of 10 mg/mL and diluted with NaCl 0.9% in a ratio of 1:1000 to yield a final concentration of 10 μg pravastatin per 1 mL carrier. The NaCl 0.9%-based carrier solution for the placebo group was prepared accordingly to include EtOH only (i.e., without pravastatin) at a concentration of 1:1000. Pregnant females were euthanized on day 15 and weights of fetuses were determined.

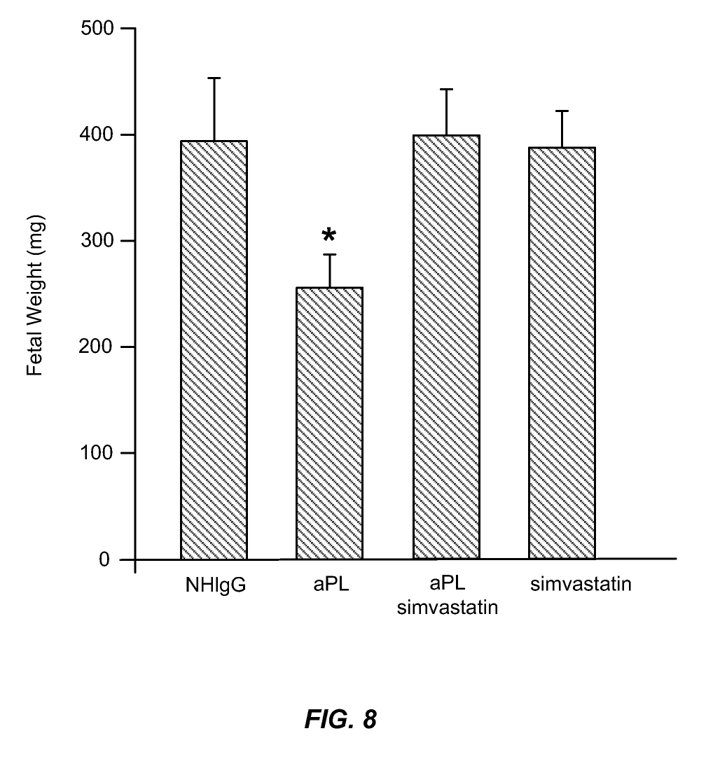

Results. The results are shown in FIG. 10. Surviving fetuses from CBA×DBA matings exhibit growth restriction. Surviving fetuses treated with pravastatin prevented IUGR.

### Example 11

### Miscarriage is Mediated by Inflammation and not the Coagulation Cascade

It is shown previously that inhibition of the coagulation cascade in a murine model of APS did not prevent pregnancy loss in this model (Girardi et al., 2004, Nat Medicine 10(11): 1222-6) and that TF modulates neutrophil activity (Redecha et al., 2007, Blood 110(7):2423-31). FIG. 4, above, shows that aPL-treated mice exhibit increased expression of PAR-2 on neutrophils. To corroborate that TF signaling through PARs is crucial for neutrophil activation and pregnancy loss, an antibody 10H10 from Dr W Ruf (Scripps Institute, LaJolla, Calif.) was used. This antibody has little impact on coagulation but potently inhibits direct TF-VIIa signaling through PAR2. On the other hand the antibody Ab 5G9 only inhibits TF-dependent coagulation. Antibodies 5G9 and 10H10 were used in tumor growth xenograft models and demonstrated that TF signaling rather than coagulation makes important contributions to tumor growth (Versteeg H H et al., 2008, Blood 111 (1): 190-9). As these antibodies are human antibodies, the study used humanized TF mice (HCV mice). HCV mice express human TF (hTF) (mTF−/−/hTF+) from a chromosome vector. These mice were generated by Dr Mackman (Pawlinski R. et al., 2007, J Thromb Haemost. 5(8):1693-700). HCV mice were treated with aPL antibodies and Abs 5G9 or 10H10 (0.5 mg/mouse, 18 hours before aPL injection).

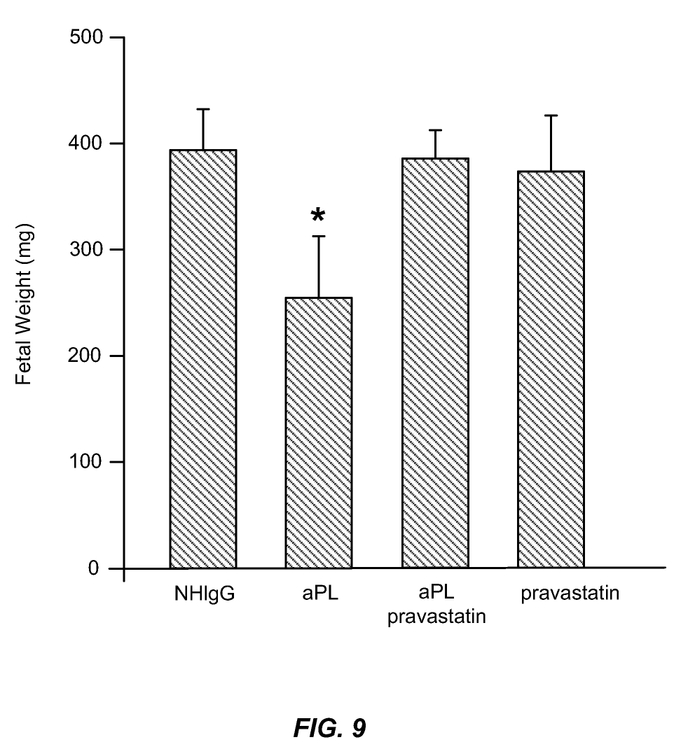

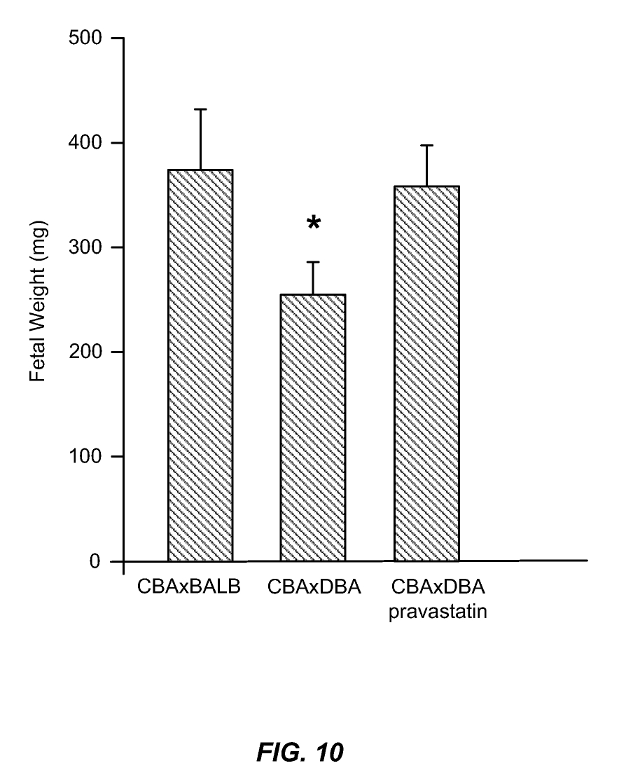

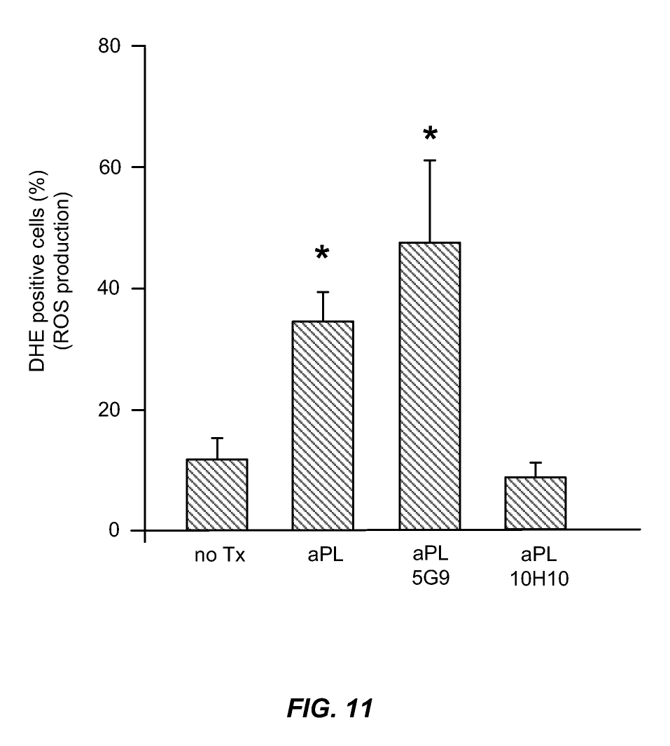

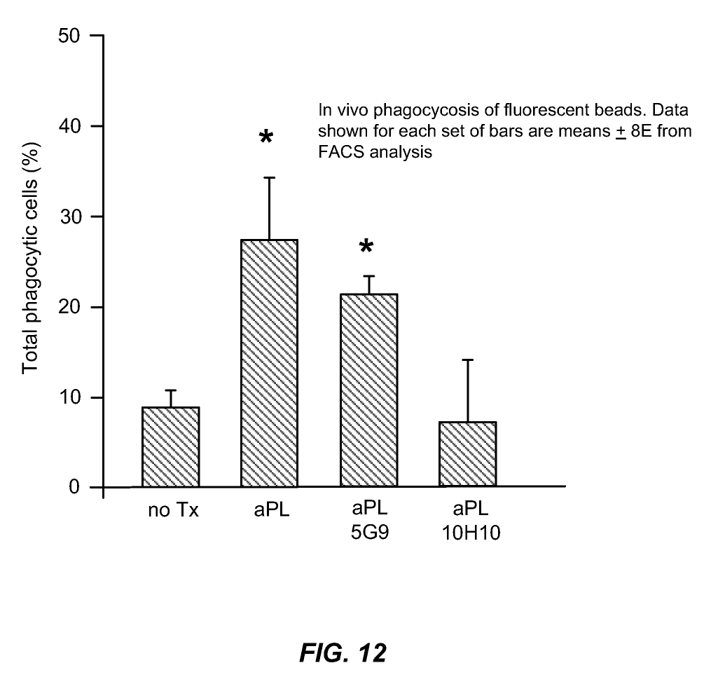

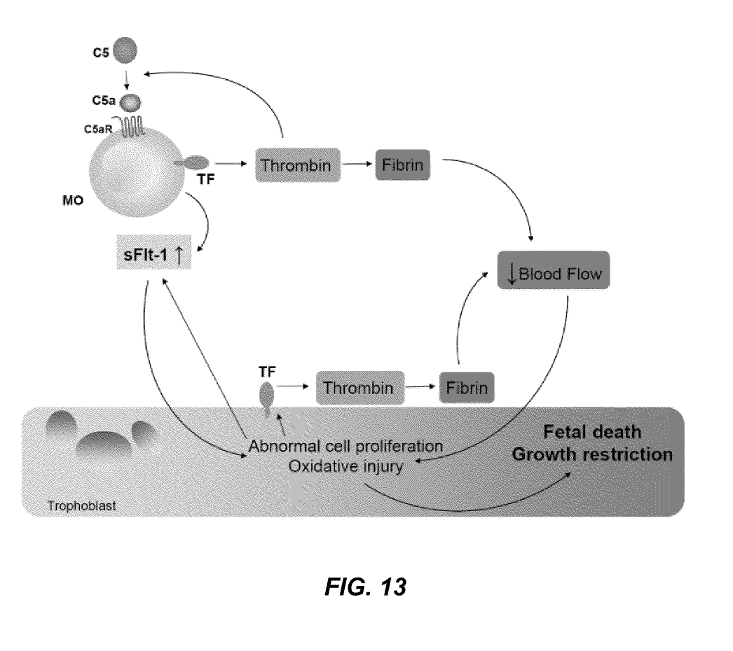

Results. Flow cytomety analysis on neutrophils shows that only antibody 10H10 which blocks direct TF-VIIa signaling through PAR2 prevented aPL-induced ROS (FIG. 11) and phagocytosis increase (FIG. 12), indicating that TF-PAR-2 interaction is a crucial mediator of aPL-induced inflammation and fetal death. Pregnancy studies could not be performed in these mice as pregnancies are not normal in these genetically modified mice, though surviving fetuses developed normally.

### Example 12

### Increased TF Staining in Deciduas and Placentas from CBA/J×DBA/2 Matings

Animals. Inbred CBA/J (H-2k), DBA/2 (H-2d) and BALB/c (H-2d) mice from The Jackson Laboratory were used. 8-10-wk-old virgin female CBA/J mice were mated with 8-14-wk-old BALB/c, or DBA/2 males. Pregnant females were sacrificed at day 7 and at day 15. The frequency of fetal resorption was calculated on day 15 as previously described (Girardi et al., 2006, J Exp Med 203:2165-2175). Fetal weights were also determined. Procedures that involved mice were approved by the Institutional Animal Care and Use Committee of the Hospital for Special Surgery and were conducted in strict accordance with guidelines for the care and use of laboratory research animals promulgated by the National Institutes of Health (NIH).

Immunohistochemistry For immunohistochemical studies in mice, frozen sections of deciduas from day 7 of pregnancy and placentas from day 15 surviving fetuses were stained for mouse TF with 1H1 antibody and for fibrin with a polyclonal rabbit antihuman fibrinogen/fibrin antibody that cross reacts with mouse fibrin (DakoCytomation, Carpinteria, Calif.). An HRP-labeled secondary antibody and DAB as substrate were used to develop the reaction. Human placentas samples, where examined, were fixed in formaldehyde and embedded in paraffin prior to sectioning for immunohistochemical studies. Antigen retrieval with boiling citrate solution pH 6 for 20 minutes was performed prior to TF immunostaining. TF was detected using FITC-conjugated mouse anti-human TF (American Diagnostica, Greenwich, Conn.). Non immune rabbit serum or non specific mouse immunoglobulin were used as negative control for rabbit and mouse primary antibodies respectively. Images were acquired using a Nikon (Japan) Eclipse E400 microscope fitted with a Nikon Digital Camera DXM 1200 (Nikon, Tokyo, Japan).

Results. In abortion-prone CBA/J×DBA/2 matings, approximately 30% of the conceptuses showed robust TF staining throughout the deciduas (d) and injured embryos at 7 of pregnancy (FIG. 14Ai). In contrast, the rest of the embryos displayed weak TF staining, comparable to embryos from control CBA/J×BALB/c matings (FIG. 14Aii). In conceptuses from control CBA/J×BALB/c matings minimal TF staining was found in the mesometrial area (FIG. 14Aii). In addition, increased TF staining was observed in the labyrinth and on the spongiotrophoblasts (sp) in day 15 placentas from the surviving growth restricted fetuses from CBA/J×DBA/2 matings (FIG. 14Aiii) when compared to placentas from fetuses in control matings (FIG. 14Aiv). The increased TF expression observed in CBA/J×DBA/matings was associated with increased fibrin staining at day 7 of pregnancy (FIG. 14Av) while minimal fibrin deposition was observed in deciduas from CBA/J×BALB/c matings (FIG. 14Avi). In addition, robust fibrin deposition was observed in the labyrinth and the spongiotrophoblast area in placentas from surviving fetuses in CBA/J×DBA/2 matings (FIG. 14Avii). Moreover, increased TF procoagulant activity was also observed in the placentas from surviving fetuses in CBA/J×DBA/2 matings when compared to CBA/J×BALB/c mice (2.1±0.3 vs 1.2±0.3 μg TF respectively, p<0.001). Despite the increased TF and fibrin expression in decidual and placental tissue in CBA/J×DBA/2 matings, no visible thrombi were found. However, thrombi can form transiently and can be rapidly lyzed.

### Example 13

### Increased TF Staining in Human Placenta from Growth Restricted Neonates

Human Placental Samples. Placentas from normal pregnancies and from intrauterine growth restricted neonates were harvested after Cesarean sections at Southern Illinois University School of Medicine. The Institutional Review Boards of SIU approved the collection and utilization of samples for research purposes. Three placentas from women with normal pregnancy and three placentas from pregnancies complicated by IUGR were studied. Women with normal pregnancies showed no medical, obstetrical or surgical complications at the time of the study and delivery of a term infant, appropriate for gestational age, without complications. Placentas from three women that gave birth to neonates with birth weight below the 10th percentile were studied in the IUGR group (Alexander et al., 1996, Obstet Gynecol 87:163-168). The placentas from neonates with IUGR were obtained from patients that developed preeclampsia (PE). Pre-eclampsia was defined in the presence of hypertension (systolic blood pressure ≧140 mmHg or diastolic blood pressure ≧90 mmHg on at least two occasions, 4 h to 1 week apart) and proteinuria (≧300 mg in a 24 h urine collection or one dipstick measurement ≧2+)

Results. To translate the findings in mice into humans, the presence of TF in human placentas was investigated. Increased TF staining was observed in the villous trophoblast cells and basement membranes in placentas from IUGR neonates (FIG. 14Biii and iv) when compared to placentas from neonates with normal body weight (FIG. 14Bi and ii).

### Example 14

### Impaired Blood Supply in Placentas from CBA/J×DBA/2 Mice

Placental Perfusion studies. Placental perfusion was examined by injecting pregnant females with 100 ul of 25 mg/ml FITC-labeled dextran (MW 2,000,000, Sigma-Aldrich, St. Louis, Mo.) via the retroorbital vein, at day 15 of pregnancy. After 15 minutes, the mice were sacrificed and the placentas removed and flash frozen. Serial frozen sections were examined and photographed under Nikon Microphot-FXA microscope (Nikon Instrument Group, Melville, N.Y.).

Results. To detect blood perfusion defects secondary to potential thrombi in placental vessels in CBA/J×DBA/2 mice, FITC-dextran was injected in the maternal circulation at day 15 of pregnancy. In control CBA/J×BALB/c matings with normal pregnancies, the fluorescent tracer accumulated in the placental labyrinth (FIG. 14Ci). In contrast, diminished blood perfusion was observed in the labyrinth of from CBA/J×DBA/2 matings (FIG. 14Cii). This observation is in accordance with the increased fibrin deposition observed in this abortion-prone CBA/J×DBA/2 mating. These data support the notion that fetal loss and growth restriction in this mouse model can be attributed to pathological activation of the coagulation cascade in placental vessels and impaired perfusion of the placenta.

### Example 15

### Increased Plasma Thrombin Antithrombin III Complex (TAT) Levels in CBA/J×DBA/2 Pregnancies

Measuring Thrombin-Anti-Thrombin Complexes. Thrombin anti thrombin III complex (TAT) was measured using a commercial ELISA kit (Enzygnost TAT; Dade Behring). Some mice from each experimental group were studied until delivery and the litter sizes were recorded at birth.

Results. Plasma thrombin-anti-thrombin III complex (TAT) levels, representing a functional state of clotting system, were studied in abortion prone and control matings. TAT plasma levels increased throughout pregnancy in CBA/J×DBA/2 mice and by day 15, CBA/J×DBA/2 mice showed TAT levels 3 times greater than those seen in the control strain combination (89±12 ng/ml in CBA/J×DBA/2 vs 31±17 ng/ml in CBA/J×BALB/c, p<0.001).

### Example 16

### Anticoagulation Protects Pregnancies in CBA/J×DBA/2 Mice

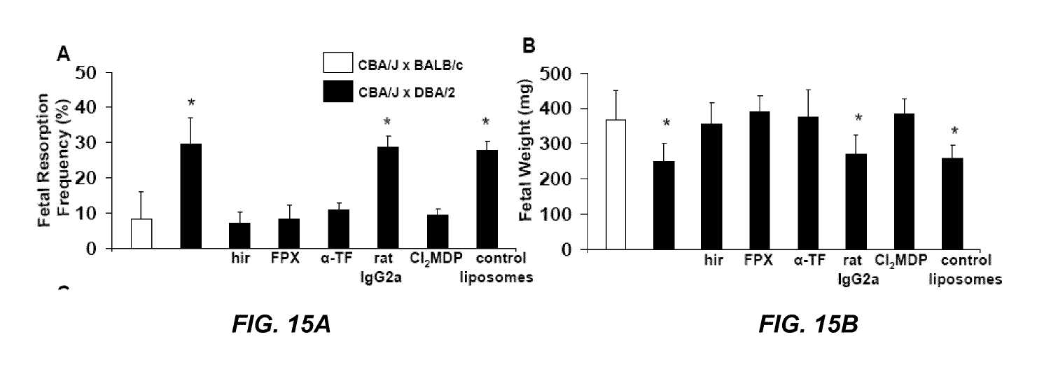

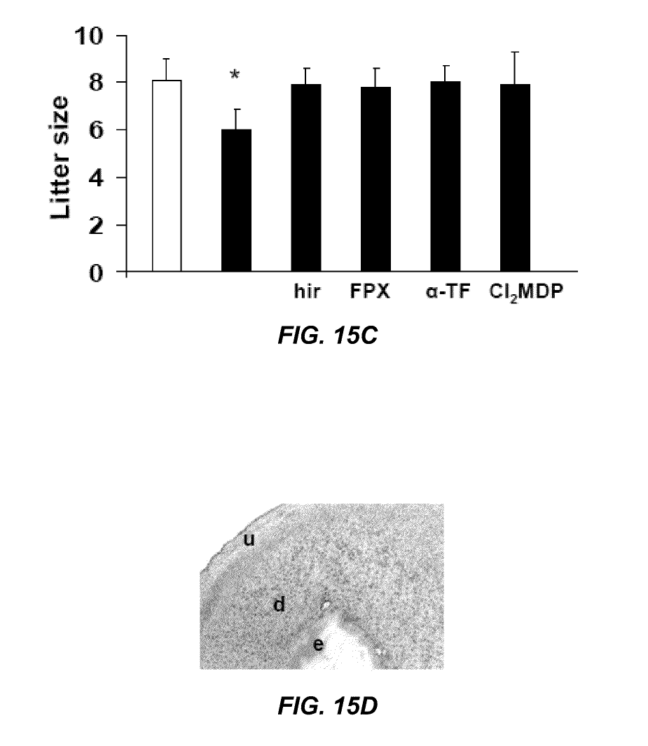

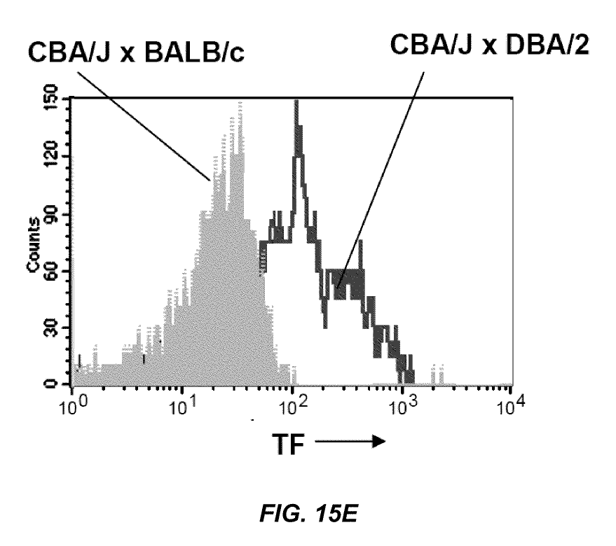

Increased coagulation seems to play a crucial role in the development of pregnancy complications in CBA/J×DBA/2 mice. To substantiate this finding, the effects of anticoagulants in CBA/J×DBA/2 mice pregnancy outcomes at day 15 were examined. As increased thrombin generation was observed in CBA/J×DBA/2 mice, the effects of direct thrombin inhibitor hirudin was examined. As expected, treatment with hirudin prevented fetal resorptions and growth restriction in CBA/J×DBA/2 mice at day 15 of pregnancy (FIGS. 15A and 15B). Fondaparinox, direct inhibitor of factor Xa, also prevented fetal loss and growth restriction in abortion-prone CBA/J×DBA/2 mice (FIGS. 15A and 15B). A group of mice was followed until the end of the pregnancy. In accordance with the data obtained at day 15 of pregnancy, DBA/2 mated CBA/J mice that showed increased fetal resorption rate at day 15, showed smaller litter sizes at birth when compared to BALB/c mated CBA/J mice (FIG. 15C). Hirudin and fondaparinox rescued pregnancies in CBA/J×DBA/2 mice increasing the litter size (FIG. 15C). That anticoagulation prevents pregnancy loss and IUGR in this model emphasizes that pathological activation of the coagulation pathway plays a role in fetal injury in the CBA/J×DBA/2 model.

### Example 17

### Blockade of TF Prevents Fetal Injury in DBA/2 Mated CBA/J Mice

TF Blockade. To block TF a group of mice were treated with i.p. injections of anti-mTF antibody 1H1 (provided by Daniel Kirchhofer, Genentech, San Francisco Calif.) (0.5 mg) (16) or an isotype-matched control antibody (rat IgG2 [symbol] Zymed Laboratories Inc, CA) (0.5 mg) on days 4, 7 and 10 of pregnancy.

Results. To assess the importance of TF in fetal death and growth restriction in CBA/J×DBA/2 mice, a rat monoclonal anti-mTF antibody 1H1 was used to inhibit TF (Redecha P et al., 2007, Blood 110:2423-2431). Administration of 1H1 was associated with a significant decrease in fetal resorption frequency (FIG. 15A) and prevention of IUGR at day 15 of pregnancy (FIG. 15B). Control antibody rat IgG2a did not affect pregnancy outcomes in CBA/J×DBA/2 mice (FIGS. 15A and 15B). A significant survival benefit for the fetuses of DBA/2-mated CBA/J mice treated with 1H1 was also observed at birth (FIG. 15C). Litter sizes in CBA/J×DBA/2 mice treated with 1H1 were not different from CBA/J×BALB/c control matings (FIG. 15C). The increased survival observed in CBA/J×DBA/2 mice treated with anti-mTF antibody 1H1 demonstrates that TF is a crucial effector in pregnancy loss in this model.

### Example 18

### Increased TF Expression on Monocytes from CBA×DBA Matings

Monocyte Depletion. To deplete monocytes/macrophages, mice were treated on day 4 of pregnancy with IV injections of dichloromethylene diphosphonate (Cl₂MDP) clodronate liposomes (Van Rooijen et al, 1994, J Immunol Meth 174:83-93.) (1 ml/100 g of body weight), which has been shown to induce the complete depletion of macrophages within 24 h (Van Rooijen et al, supra). Repeat injections of 0.5 ml/100 g Cl₂MDP clodronate liposome suspension were made on day 8 and 12 of pregnancy. Monocyte depletion in peripheral blood and decidual tissue persisted until the end of the studies. Mice receiving phosphate-buffered saline (PBS) containing liposomes served as controls.

Determining TF Expression. TF expression on peripheral blood monocytes was analyzed by 2-color fluorescence-activated cell sorting (FACS). Heparinized whole blood obtained at day 7 from DBA/2 or BALB/c mated CBA/J females was stained with fluorescein isothiocyanate (FITC)-labeled anti-mouse F4/80 for FL1 (BD Biosciences Pharmingen) to identify monocytes and biotinylated rat anti-mTF antibody 1H1 and streptavidin-PerCP (BD Biosciences Pharmingen) as the FL3 fluorochrome. Red cells were lysed with ACK buffer (Redecha et al., supra). Preparations were then incubated with SA-PerCP and analyzed by FACS using a FACscan (BD Biosciences Pharmingen).

Results. Because monocytes have shown to be important mediators of fetal damage in this model (Girardi et al., 2006, J Exp Med 203:2165-2175; 23-Clark et al., 1998, J Immunol 160:545-549.) and these cells have been shown to express TF in response to C5a (Clark et al., 1998, J Immunol 160:545-549.), the contribution of monocytes to the generation of decidual TF in CBA/J×DBA/2 was investigated. Monocytes depletion was achieved using Cl₂MDP clodronate liposomes (Van Rooijen and Sanders, 1994, J Immunol Meth 174:83-93). Neither pregnancy complications (FIGS. 15A and 15B) nor increase in decidual TF expression was observed in CBA/J×DBA/2 mice lacking monocytes (FIG. 15D). CBA/J×DBA/2 mice treated with Cl₂MDP clodronate showed minimal TF staining in deciduas at day 7 of pregnancy (FIG. 15D). Macrophage depletion also increased the number of surviving fetuses as indicated by the bigger litter sizes at birth (FIG. 15C).

Given that monocytes are critical effectors of fetal death, and that TF is required for pregnancy complications in CBA/J×DBA/2 mice, monocytes from CBA/J×DBA/2 mice at day 7 of pregnancy were tested for increased TF expression. Monocytes from CBA/J×DBA/2 mice were 32±9% positive for TF (FIG. 15E) compared to 6±3% of the monocytes from CBA/J×BALB/c mice (p<0.005). Less than 1% of the monocytes showed non-specific binding when incubated with isotype control antibody rat IgG2a.

### Example 19

### Increased Placental Oxidative Stress in CBA/J×DBA/2 Matings

STAT-8 Measurements. Isoprostane 8-iso-prostaglandin F2a (STAT-8) was measured as a marker for oxidative stress. Placental tissue was harvested at day 15 of pregnancy and homogenized in 9 volumes of 0.1 M Tris pH 7.4 containing 1 mM EDTA and 10 μM indomethacin and stored at −80° C. in the presence of 0.005% BHT before assayed for free 8-isoprostane using a STAT-8-Isoprostane EIA kit (Cayman Chemical Company, Ann Arbor, Mich.).

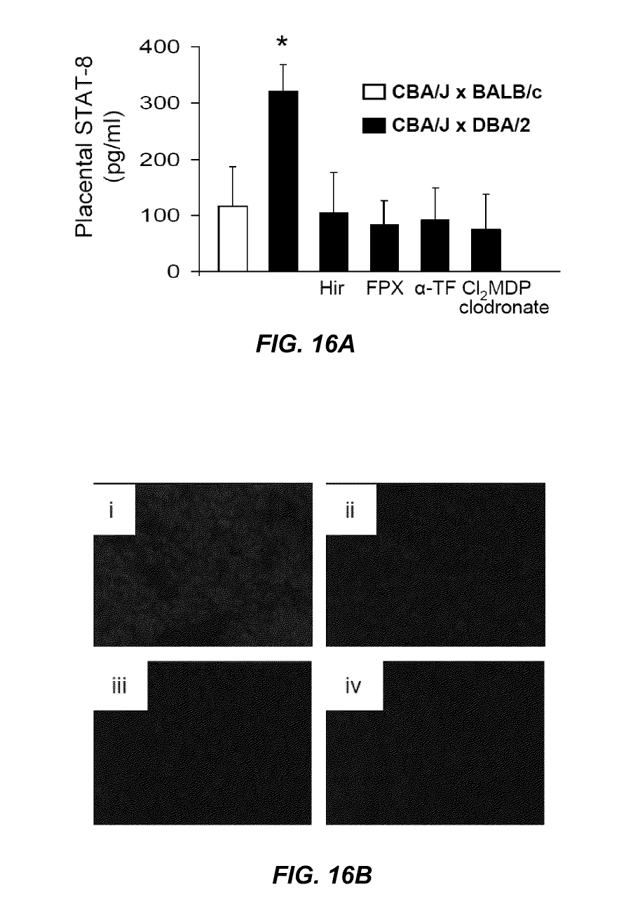

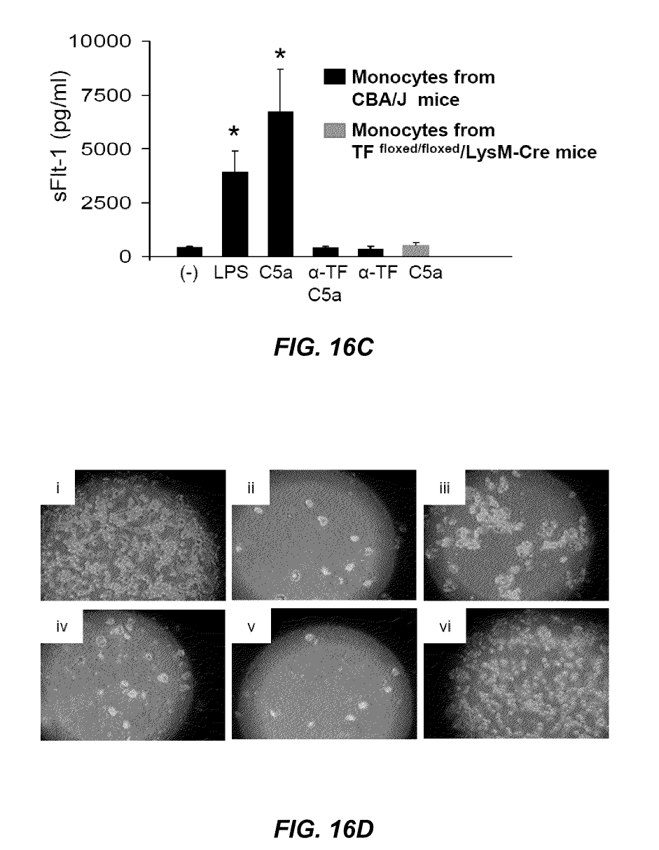

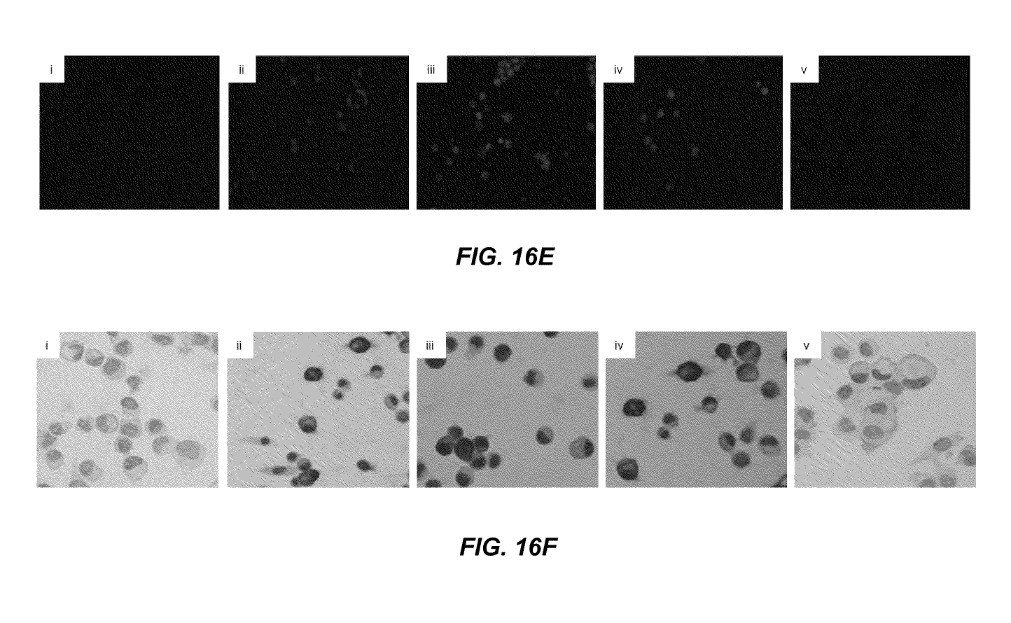

Results. There is mounting evidence that oxidative stress in placental tissue plays a pivotal role in the development of placental-related diseases like fetal death and IUGR (El-Far et al., 2007, Clin Chem Lab Med 45:879-883). Isoprostane 8-iso-prostaglandin F2a (STAT-8) is a marker for oxidative stress in vivo. STAT-8 is also a potent vasoconstrictor, activates platelets, induces derangement of endothelial cells (Patrono et al., Arterioscler Thromb Vasc Biol 17:2309-2315.) and reduces trophoblast invasion in vitro (Staff et al., 2000, Hypertension 35:1307-13). Increased STAT-8 levels was observed in day 15 placentas from DBA/2 mated CBA/J females when compared to uneventful pregnancies in control matings (FIG. 16A). These data are in accordance with studies showing increased STAT-8 levels in women with pregnancy complications (Barden et al., 1996, Clin Sci 91:711-718; Walsh et al., 2000, FASEB J 14: 1289-1296.). In addition to the increased STAT-8 levels, placentas from CBA/J×DBA/2 matings exhibited increased superoxide production measured as dihydroethidium (DHE)-derived fluorescence at microscopy (FIG. 16Bi) when compared to CBA/J×BALB/c mice (FIG. 16Bii). These results indicate there is a correlation between pregnancy complications and placental oxidative stress injury.

Treatment with antibody anti-TF 1H1 prevented STAT-8 increase (FIG. 16A) and superoxide production in CBA/J×DBA/2 mice (FIG. 16Biii). Anticoagulation with hirudin or fondaparinox also prevented STAT-8 increase in CBA/J×DBA/2 mice (FIG. 16A). In addition, monocyte depletion with CL₂MDP clodronate liposomes, that protected pregnancies, also prevented placental STAT-8 increase (FIG. 16A) and placental superoxide production in CBA/J×DBA/2 mice (FIG. 16Biv). These data suggest that TF and monocytes are essential participating factors in placental oxidative damage and pregnancy complications in this model.

### Example 20

### Monocytes do not Release sFlt-1 in Response to C5a in the Absence of TF

Protocol. Mice with a selective deletion of the TF gene in myeloid cells (TF^{floxed/floxed}/LysM-Cre mice) (Redecha et al., supra), were used for isolation of monocytes to study sVEGFR-1 production in vitro. Blockade of TF was carried out as in Example 17 by i.p. injections of anti-mTF antibody 1H1.

Results. It has previously been shown that complement component C5a triggers release of sFlt-1 by monocytes in vitro (Girardi et al., J Exp Med 203:2165-2175). LPS also stimulates sFlt-1 release from monocytes (FIG. 16C). Blockade of TF with antibody 1H1 or genetic deletion of TF on monocytes (TF^{floxed/floxed} LysM-Cre mice) prevented sFlt-1 release in response to C5a (FIG. 16C), suggesting that TF expression on monocytes is required for sFlt-1 release. In addition, in vivo experiments show that TF blockade diminished plasma sFlt-1 levels in CBA/J×DBA/2 mice at day 7 of pregnancy (sFlt-1 (pg/ml): 16890±6745 in CBA/J×DBA/2+1H1 (n=7) vs 29780±8760 in untreated DBA/2 mated CBA/J mice (n=10), p<0.01).

### Example 21

### Reduced Cell Proliferation, Increased Oxidative Injury and Increased TF Expression in Trophoblasts Incubated with sFlt-1

Protocol. SM9-1 trophoblast cells, derived from a gestational day 9 Swiss-Webster mouse placenta (SM9-1) (Bowen et al., Biol Reprod 60:428-434.), were incubated with the supernatants from monocytes incubated with C5a containing high levels of sFlt-1 (Girardi et al., 2006, J Exp Med 203:2165-2175). Some SM9-1 cells were incubated in the presence of increasing amounts of mouse sFlt-1 (R&D Systems) (1000, 2000, 4000 and 8000 pg/ml). After 72 hours, cell proliferation was evaluated by light microscopy. Some SM9-1 cells were incubated with pravastatin (5 μg/ml) for 3 hours prior to incubation with sFlt-1. Mouse VEGF (500 pg/ml) (R&D systems) was added to some SM9-1 incubated with sFlt-1 or monocytes supernatants. Immunohistochemical identification of TF with 1H1 and superoxide production by DHE was performed on the SM9-1 cells after 72 hours of culture.

Results. sFlt-1 is significantly increased in plasma from DBA/2 mated CBA/J mice and is released by monocytes incubated with C5a (Girardi et al., supra). Since sFlt-1 is a potent antiangiogenic molecule that sequesters circulating VEGF (Girardi et al., supra; Maynard et al., 2003, J Clin Invest 111:649-658.31), how sFlt-1 affects trophoblast proliferation in vitro was studied. SM9-1 cells incubated with monocytes supernatants (FIG. 16Dii) or sFlt-1 (FIG. 16Diii, iv and v) showed diminished cell proliferation compared to cells incubated with culture media only (FIG. 16Di). The antiproliferative effect of sFlt-1 on trophoblasts was dose response (FIG. 16Diii-iv). Addition of VEGF to the trophoblasts incubated with monocytes supernatants or sFlt-1 restored cell proliferation (FIG. 16Dvi). These data indicate that sFlt-1 by quenching VEGF causes abnormal proliferation of trophoblasts and may be the cause for abnormal placentation and poor pregnancy outcomes in CBA/J×DBA/2.

In addition to inhibition of proliferation, trophoblast cells incubated with supernatants of monocytes incubated with C5a (FIG. 16Eii) and sFlt-1 (FIG. 16Eiii and iv) exhibited increased superoxide production measured by DHE. Addition of VEGF restored cell growth and prevented oxidative damage in trophoblasts (FIG. 16Ev). Trophoblasts incubated with media only showed abundant cells and weak DHE fluorescence (FIG. 16Ei). Increased TF staining was also found in SM9-1 trophoblasts incubated with supernatants from monocytes or with sFlt-1 (FIG. 16Fiii and iv). Addition of VEGF prevented TF expression (FIG. 16Ev). These data suggest that the inhibition of trophoblast proliferation by sFlt-1 is mediated by VEGF and is associated with increased oxidative stress and increased TF expression.

### Example 22

### Pravastatin Prevented Pregnancy Loss and Placental Oxidative Stress, and Restored NO Levels in CBA/J×DBA/2 Mice

Protocol. To determine the effect of pravastatin treatment on pregnancy loss and oxidative stress, group of mice was treated with pravastatin (Sigma Chemicals, St Louis, Mo.; 5 μg/mouse, IP) from day 4 to 15 of pregnancy. Pravastatin was directly dissolved in sterile PBS. Blood samples were obtained from pregnant females from days 1 to 15 of pregnancy from the submandibular vein. Nitric oxide (NO) was measured by a calorimetric method (R&D Systems). Plasma sVEGFR-1 was measured with a commercial ELISA kit (R&D Systems).

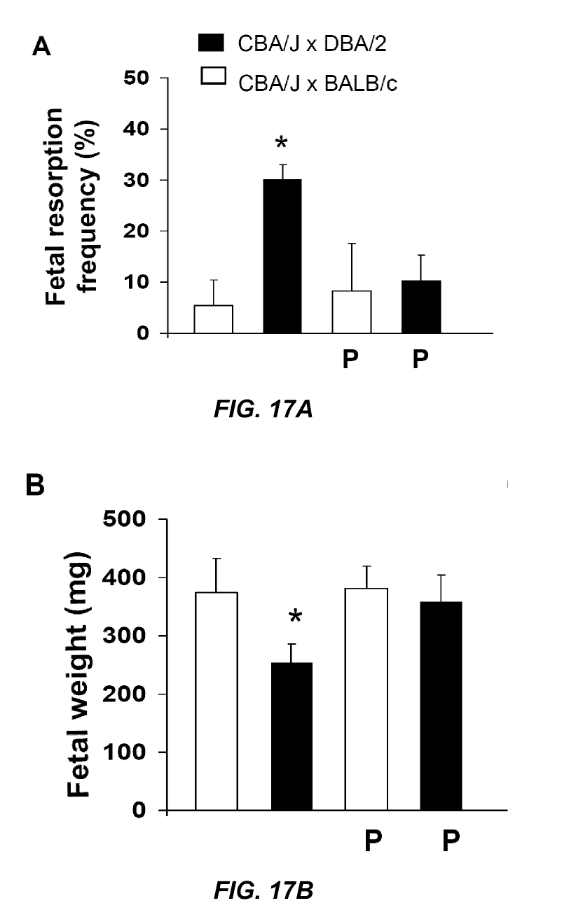

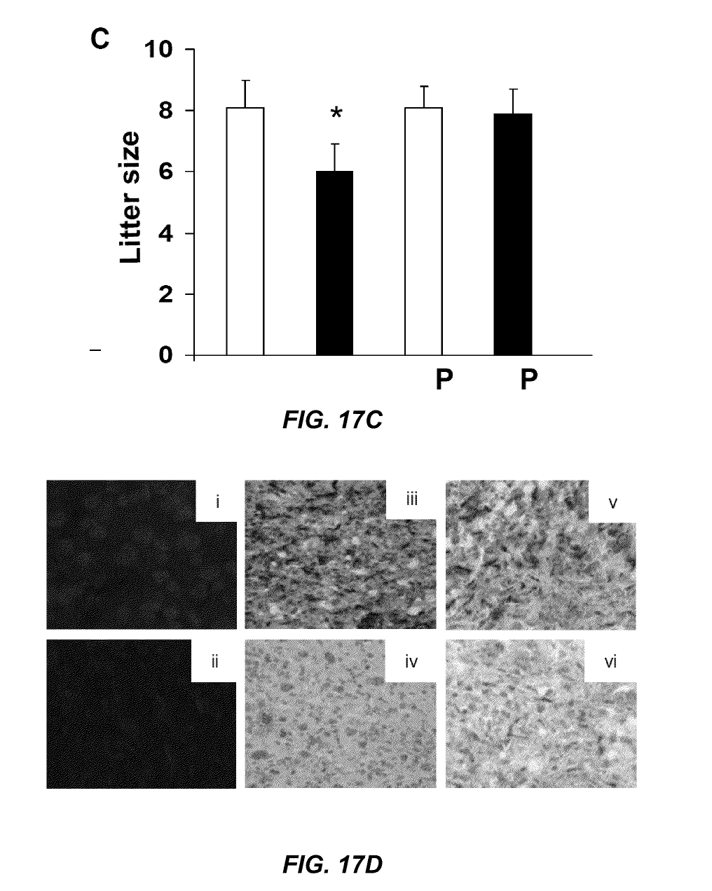

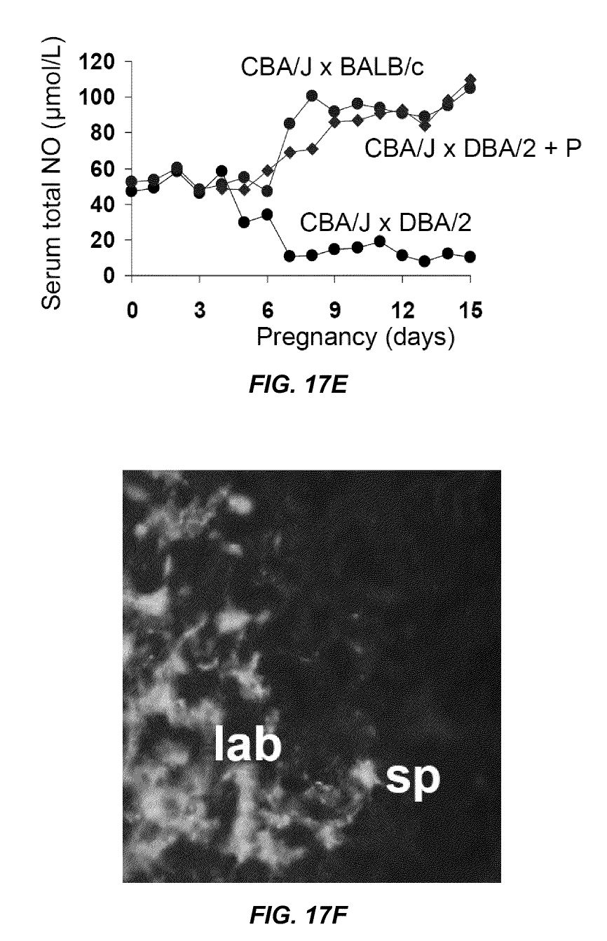

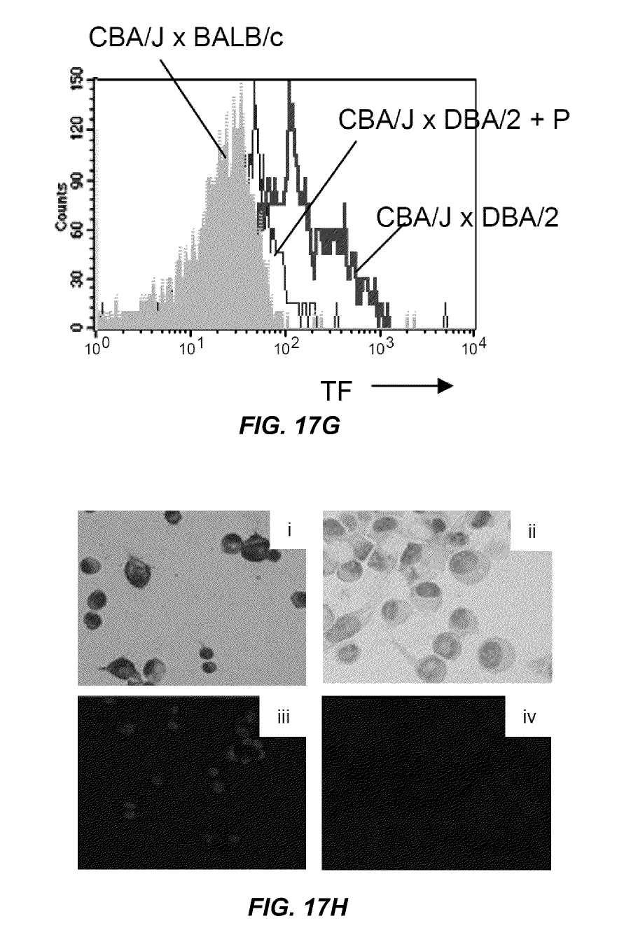

Results. Statins are postulated to inhibit inflammation and coagulation (Rosenson et al., 1998, JAMA 279:1643-1650; Takemoto et al., 2001, Arterioscler Thromb Vasc Biol 21:1712-1719) and to reduce TF expression and activity in blood monocytes and neutrophils (Redecha et al., supra; Wei et al., 2007, Eur J Med Res 12:216-221; Steiner et al., 2005, Circulation 111: 1841-1846). Since TF is a crucial mediator in pregnancy complications observed in CBA/J×DBA/2 mice, the ability of pravastatin to prevent pregnancy loss in this model of spontaneous miscarriages was studied. In accordance to our hypothesis, pravastatin prevented fetal loss (FIG. 17A) and IUGR (FIG. 17B) in DBA/2 mated CBA/J mice at day 15 of pregnancy. The beneficial effects of pravastatin on pregnancy outcomes in CBA/J×DBA/2 mice were also found at birth (FIG. 17C). Pravastatin also prevented oxidative stress (FIG. 17Dii) and decreased TF expression (FIG. 17Div) and fibrin deposition (FIG. 17Dvi) in placentas from CBA/J×DBA/2 mice when compared to untreated CBA/J×DBA/2 mice (FIG. 17Di, iii and iv).

In normal CBA/J×BALB/s mating, plasma NO levels increase gradually along pregnancy (FIG. 17E). In contrast, NO levels do not increase in CBA/J×DBA/2 mice (FIG. 16E). Plasma NO levels in CBA/J×DBA/2 mice treated with pravastatin were not different from control matings with good pregnancy outcomes (FIG. 17E). Consistent with the finding that pravastatin prevented plasma NO diminution and inhibited placental fibrin deposition, pravastatin restored placental blood flow in CBA/J×DBA/2 mice (FIG. 17F). By preventing the diminution of vasodilator molecule NO and inhibiting fibrin deposition, pravastatin restores placental blood flow and rescues pregnancies in CBA/J×DBA/2 mice.

### Example 23

### Pravastatin Prevented sFlt-1 Release and TF Expression by Monocytes

Protocol. Splenocytes were prepared from non pregnant CBA/J female mice and incubated for 4 h in culture medium supplemented with 10% inactivated fetal bovine serum (FBS). Non adherent cells were removed and adherent cells (>95% peroxidase-positive) were incubated in culture medium with the addition of 10 nM mouse recombinant C5a. A group of adherent monocytes was treated for 3 hours with pravastatin (5 μg/ml) prior to incubation with C5a. After 4 h of incubation with C5a, culture supernatants were collected and analyzed for sVEGFR-1 by ELISA (R&D Systems).

Results. It is shown herein that in response to C5a, monocytes release high amounts of sFlt-1. Monocytes pre-incubated with pravastatin release small amounts of sFlt-1 in response to C5a (sFlt-1 (pg/ml):C5a (n=5): 6749±1927 vs 387±78 in C5a+pravastatin (n=4), p<0.01). Immunocytochemical staining revealed minimal TF staining in monocytes treated with C5a that have been previously incubated with pravastatin (data not shown). In addition, FACs analysis showed that pravastatin prevented TF expression on monocytes in CBA/J×DBA/2 mice ((% TF positive monocytes: 32.5±9 in CBA/J×DBA/2 mice vs 10±4 in CBA/J×DBA/2+pravastatin, p<0.01, n=5-6) (FIG. 17G). These data reinforce the idea that TF expression on monocytes is an essential factor in the pathogenesis of fetal injury in CBA/J×DBA/2 mice. In addition, pravastatin treatment diminished plasma sFlt-1 levels in CBA/J×DBA/2 mice at day 7 of pregnancy (sFlt-1 (pg/ml): 18650±5680 in CBA/J×DBA/2+pravastatin (n=6) vs 29780±8760 in untreated DBA/2 mated CBA/J mice (n=10), p<0.01).

### Example 24

### Pravastatin Prevented Oxidative Stress and TF Expression in Mouse Trophoblasts

Protocol. SM9-1 trophoblast cells were prepared as described in Example 21. Isolated SM9-1 cells were incubated in the presence of increasing amounts of mouse sFlt-1 (R&D Systems) (1000, 2000, 4000 and 8000 pg/ml). After 72 hours, cell proliferation was evaluated by light microscopy. SM9-1 cells were also incubated with pravastatin (5 μg/ml) for 3 hours prior to incubation with sFlt-1. Immunohistochemical identification of TF with 1H1 and superoxide production by DHE was performed on the SM9-1 cells after 72 hours of culture as described above.

Results. As shown herein, mouse trophoblasts incubated with sFlt-1 exhibited growth inhibition, increased TF expression and increased superoxide production. Incubation of trophoblasts with pravastatin prior to sFlt-1 incubation restored cell growth, prevented TF expression (FIG. 17Hii), and prevented oxidative damage (FIG. 17Hiv).

### Example 25

### Statins are Safe and Non-Teratogenic

Epidemiological data collected by other investigators to date suggest statins are not major teratogens. Pregnancy outcomes after maternal exposure to simvastatin showed no evidence of increase congenital anomalies (Pollack P S et al, 2005, Birth Defects Res A Clin Mol Teratol. 73(11):888-96). Other studies suggest that statins in general do not have adverse effects on the developing fetus during pregnancy (see, e.g., Kazmin et al., 2007, “Risks of Statin Use During Pregnancy:A Systematic Review,” J Obstet Gynaecol Can. 29(11):906-8.

Pravastatin may provide additional benefits, as it is not believed to reach the embryo, and thus is not expected to down-regulate cholesterol synthesis or other metabolic intermediates (see, e.g., Edison et al., 2005, Am J Med Genet A. 135(2):230-231).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of preventing or reducing risk of pregnancy loss or miscarriage in a patient, comprising administering to a patient who is pregnant or planning to become pregnant an amount of a tissue factor (“TF”) expression inhibitor compound effective to inhibit TF expression.

2. The method of claim 1 in which the patient has increased number of TF-positive neutrophils.

3. The method of claim 1 in which the patient has increased number of TF-positive monocytes.

4. The method of claim 1 in which the patient has elevated levels of TF in blood.

5. The method of claim 1 in which the patient has elevated levels of TF in chorionic villus.

6. The method of claim 1 in which the TF-expression inhibitor compound is a statin, or a salt thereof.

7. The method of claim 1 in which the TF-expression inhibitor compound is simvastatin or a salt thereof.

8. The method of claim 1 in which the TF-expression inhibitor compound is pravastatin or a salt thereof.

9. The method of claim 1 in which the TF-expression inhibitor compound is administered at a dosage in the range of about 10 to 100 mg per day.

10. The method of claim 1 in which the patient shows no clinical symptoms of preeclampsia.

11. The method of claim 1 in which the patient is pregnant.

12. The method of claim 1 in which the patient is planning to become pregnant.

13. The method of claim 1 in which the patient has previously miscarried.

14. The method of claim 1 in which the patient has suffered multiple previous miscarriages.

15. The method of claim 1 in which the patient exhibits or suffers from APS.

16. A method of diagnosing a patient for increased risk of pregnancy loss or miscarriage, comprising assessing the patient for increased presence of tissue factor (TF).

17. The method of claim 16 in which the increased presence of TF is determined for population of neutrophils.

18. The method of claim 16 in which the increased presence of TF is determined for population of monocytes.

19. The method of claim 16 in which the increased presence of TF is determined for blood.

20. The method of claim 16 in which the increased presence of TF is determined for chorionic villus.

21. The method of claim 16 in which the increased presence of TF is determined over a period of time.

22. The method of claim 16 in which the increased presence of TF is determined over a period of time.

23. The method of claim 16 in which the patient is pregnant.

24. The method of claim 16 in which the patient is planning to become pregnant.
